(19) United States
(12) Patent Application Publication
(10) Pub. No.: US 2014/0194949 A1
(43) Pub. Date: Jul. 10, 2014

(54) Multiplex Electrodes for Applying Transcutaneous Interferential Current

(71) Applicant: Brian D. Wichner, Otter Rock, OR (US)

(72) Inventor: Brian D. Wichner, Otter Rock, OR (US)

(21) Appl. No.: 13/734,918

(22) Filed: Jan. 4, 2013

(51) Int. Cl. A61N 1/36

(52) U.S. Cl. CPC A61N 1/36003
USPC 607/48

(57) ABSTRACT

Subject matter includes a method comprising: generating a first electrical signal having a time-varying waveform; generating a second electrical signal having a time-varying waveform, wherein a sum of the first electrical signal and the second electrical signal comprises a target electrical signal having a waveform to generate substantial motor movement of the one or more muscles in the patient; and applying the first electrical signal via a first electrode and the second electrical signal via a second electrode to a particular location on the patient, wherein the first electrode and the second electrode are on a single electrode pad.

### BACKGROUND

1. Field

Subject matter disclosed herein relates to an apparatus and method for providing electrotherapeutic signals to a patient.

2. Information

A number of techniques for treating a patient or detecting a physical condition of a patient may involve applying electrical energy via electrodes in contact with the patient. Such electrodes may comprise pads having an adhesive (or a water-activated adhesive) to temporarily affix the pads to a portion of a patient. For example, a transcutaneous electrical nerve stimulation (TENS) device may apply electric current to a patient via electrode pads to stimulate nerves of the patient for therapeutic purposes. In another example, muscle loss of a patient may be determined using electric impedance myography (EIM), which may measure resistance of a muscle to an electrical current by passing an amount of current through the muscle using electrodes.

Electrical current of electrodes applied to a patient may flow through a number of characteristic regions of the patient. For example, current from a first electrode applied to skin may flow through the skin and subsequently, in varying degrees, through plasma, fascia, muscle tissue, bones, ligaments, and/or organs, and out through skin to a second electrode. Such individual characteristic regions have particular electrical properties, such as electrical resistance, impedance, capacitance, and so on.

### BRIEF DESCRIPTION OF THE FIGURES

Non-limiting and non-exhaustive embodiments will be described with reference to the following figures, wherein like reference numerals refer to like parts throughout the various figures unless otherwise specified.

FIG. 1 is a cross-sectional schematic diagram illustrating electrodes for applying one or more electrical signals to a portion of a patient, according to an embodiment.

FIG. 2 is a schematic diagram illustrating an electronic device for electrical stimulation, according to an embodiment.

FIGS. 3 and 4 show example signal waves plotted as magnitude of voltage or current versus time, according to embodiments.

FIG. 5 is a cross-sectional schematic diagram illustrating an electrical component analogue corresponding to a portion of a patient, according to an embodiment.

FIG. 6 is a schematic diagram illustrating electrical stimulation in a portion of a patient, according to an embodiment.

FIG. 7 shows a superposition of two sinusoidal waveforms having dissimilar frequencies, according to an embodiment.

FIG. 8 is a schematic diagram illustrating electrical stimulation in a portion of a patient, according to another embodiment.

FIG. 9 is a cross-sectional schematic diagram illustrating an electrical component analogue corresponding to a skin portion of a patient, according to an embodiment.

FIG. 10 is a cross-sectional schematic diagram illustrating an electrical component analogue corresponding to a portion of a patient, according to another embodiment.

FIG. 11 is a schematic diagram illustrating electrical stimulation in a portion of a patient, according to yet another embodiment.

FIG. 12 is a schematic diagram illustrating an electronic device and double-capacitive transcutaneous electrode pads for electrical stimulation, according to an embodiment.

FIG. 13A is a perspective view of a double-capacitive transcutaneous electrode pad, according to an embodiment.

FIG. 13B is a cross-sectional view of a double-capacitive transcutaneous electrode pad, according to an embodiment.

FIGS. 13C-13H are bottom views of example embodiments of various configurations of double or multi-capacitive transcutaneous electrode pads.

FIGS. 14-19 are plots of characteristics for first and second electrical signals and their superposition as a function of time, according to embodiments.

FIG. 20 is a flow diagram of a process for applying electrical signals to a patient for stimulating one or more muscles of the patient, according to an embodiment.

FIG. 21 shows superpositions of two sinusoidal waveforms having dissimilar frequencies and a schematic diagram illustrating electrical stimulation in a portion of a patient, according to an embodiment.

FIG. 22 is a schematic block diagram illustrating a system for generating electrical signals to apply to a patient for stimulating one or more muscles of the patient, according to an embodiment.

FIG. 23 is a schematic block diagram illustrating a system for performing a process for applying electrical signals to a patient for stimulating one or more muscles of the patient, according to another embodiment.

FIG. 24 is a schematic block diagram illustrating a computer system, according to an embodiment.

### DETAILED DESCRIPTION

Reference throughout this specification to “one embodiment” or “an embodiment” means that a particular feature, structure, or characteristic described in connection with the embodiment is included in at least one embodiment of claimed subject matter. Thus, the appearances of the phrase “in one embodiment” or “an embodiment” in various places throughout this specification are not necessarily all referring to the same embodiment. Furthermore, the particular features, structures, or characteristics may be combined in one or more embodiments.

Impedance may refer to the opposition that a path of electrical current presents to the passage of the current if a voltage is applied. For example, in quantitative terms, impedance may comprise a complex ratio of the voltage to the current. Impedance (e.g., for time-varying electrical signals) may comprise an extension of the concept of resistance (e.g., non-time-varying electrical signals), and may include both magnitude and phase, unlike resistance, which may only include magnitude. In situations involving time-varying electrical signals, mechanisms in addition to normal resistance (e.g., ohmic resistance for non-time-varying electrical signals) may impede flow of current. Such mechanisms may comprise induction of voltages in conductors self-induced by magnetic fields of currents (inductance), and electrostatic storage of charge induced by voltages between conductors (capacitance). Impedance based, at least in part, on these two effects may collectively be referred to as reactance and forms an imaginary part of complex impedance whereas resistance forms a real part, for example.

The terms “resistance” and “impedance” are used herein interchangeably to mean the same thing unless used in the context of a sentence that indicates otherwise. For example, “resistance” means impedance that may comprise an inductive reactance, capacitive reactance, and/or ohmic resistance. On the other hand, “impedance” may mean ohmic resistance and may or may not include inductive reactance and/or capacitive reactance. Again, a context or description of a sentence or portion of text in which such terms are used may indicate one meaning over another meaning. The term “resistance” may comprise inductive reactance, capacitive reactance, and/or ohmic resistance. If “resistance” is intended to exclude inductive reactance and/or capacitive reactance then the term “ohmic resistance” is used.

The term “patient” is recited in examples herein. A patient need not comprise a subject who is ill, sick, or stricken with any particular medical condition. A patient may comprise a medical patient, a dental patient, a physical therapy client, a massage client, or one who has treatment or a physical process applied to any portion of their body for any of a number of reasons. Unless otherwise described, a patient may comprise human, animal, fish, reptile, bird, and so on. In some embodiments, a patient may comprise abiotic systems or material, such as liquid, mineral, plastics, etc., although example embodiments are directed to biotic systems. For example, embodiments of techniques described may be applied in cases where a patient is human or where a patient is a fish or animal, and claimed subject matter is not limited in this respect. To describe a particular implementation, techniques may be applied to diagnose a physical or mental condition (e.g., muscle mass, cancer, blood chemistry, brain disorder, and so on) of a human patient. In another implementation, techniques may be applied to perform research regarding any of a number of physical parameters of various aquatic species. In the latter implementation, the “patient” may comprise a particular aquatic specimen. Other implementations may involve animals, and so on. Accordingly, though the following descriptions may indicate a human patient, claimed subject matter is not limited in this respect. Further, “patient” need not comprise a person undergoing or seeking medical treatment or diagnoses. For example, a patient may comprise any person (e.g., or other species, as described above) to which an electric waveform may be applied for any reason.

The term “therapist” is intended to include any operator of devices or an applicator of electrical signals, and is not limited to professional practitioners. Thus, a therapist may comprise any person, including a patient.

Biological elements of a subject may comprise any portion or combination of portions of the subject, such as skin, muscle tissue, organs, normal or cancer cells, blood, ligaments, tendons, bones, scar tissue, and so on. Such biological elements may be microscopic or macroscopic. Such biological elements may be in any type of condition, such as healthy or normal, damaged or injured, deteriorated, inflamed, and so on.

Injured tissue may result from force transferring to a portion of a patient not designed to absorb the force. An inability to absorb force properly may be due to an inability to control muscles properly. Applying electrical energy to a patient may allow a therapist to search a patient's body for a source of an injury, thus allowing the therapist to know where on the patient to perform therapy, for example.

Applying electrical energy to a patient may increase permeability of muscle tissues of a patient. Often, injuries may not efficiently heal because blood cannot flow to an injured area. Applying electrical energy to a patient may break bonds holding scar tissue together and allow the scar tissue to be flushed away with increased blood flow. With less scar tissue surrounding an injured area, more blood may be able to flow to an injury site and shorten healing time, for example.

Rate of healing may depend on an amount of blood flow to an area of injury. Applying electrical energy to a patient may increase blood flow. Increasing blood flow may allow the body of a patient to bring more protein to an area of injury for repair and for flushing out toxins associated with inflammation and scar tissue, for example.

In some embodiments, applications of electrical energy (e.g. for muscle stimulation, cellular regeneration, physical or mental diagnosis, and so on) may involve a power source, a signal generator, at least two electrodes, and leads (e.g., cables, wires, conductors, and so on). Electrical energy application may comprise transcutaneous application, involving leads or electrodes on skin of a patient, for example. Electrical energy may comprise a waveform having a number of parameters, including one or more frequencies, waveshapes, voltage/current amplitude, phase shift, energy, power, zero-offset, slope, and so on.

In an embodiment, a device, which may comprise a medical device, may be used to apply one or more electrical waveforms (e.g., signals) to a patient. A waveform may comprise an electrical signal that may be used for therapy, treatment, or diagnostics of one or more medical conditions of a patient, for example. Waveforms may have a number of parameters, as described above. Different waveforms may be used to treat different patients, to treat different medical conditions, to perform different treatments at various stages of application to a patient, to detect medical conditions of different portions of a patient, to measure different medical conditions of a patient, and so on.

Electrical signals may comprise any of a number of forms. For example, a signal may comprise analog or digital electronic signals transmitted in a conductor or transmitted wirelessly, may comprise analog or digital electronic signals stored in a storage medium such as a memory device, may comprise analog electronic signals transmitted in cables or conductors (e.g., to/from a patient), may comprise a digital or analog code readable by a processor to generate a digital or analog electronic signal, and so on.

In an embodiment, a method or technique may be used to transcutaneously apply electrical energy to a patient. For example, transcutaneously applying electrical energy to a patient may electrically stimulate one or more muscles in the patient. In an implementation, a system or device may perform such a method or technique. Such a method or technique may comprise generating a first electrical signal and a second electrical signal having time-varying waveforms. The sum of the first electrical signal and the second electrical signal may comprise a target electrical signal having a waveform to generate or stimulate substantial motor movement of one or more muscles in a patient, for example, though claimed subject matter is not limited to a target electrical signal that generates muscle movement. For example, a target waveform may be used to improve blood circulation, provide massage therapy, or modify cellular processes in a patient. The method or technique may further comprise applying the first electrical signal via a first electrode and the second electrical signal via a second electrode to a particular location on the patient, wherein the first electrode and the second electrode may be on a single electrode pad. The single electrode pad may be applied on skin of the patient so that the first electrode and the second electrode are electrically separated by capacitance of the skin based, at least in part, on electrical properties (e.g., impedance, capacitance, and so on) of dermis and subcutis of the skin, as described below. For example, placing such a single electrode pad on skin of a patient may allow the first electrical signal and the second electrical signal to combine (superpose) below the skin to form the target electrical signal. In one implementation, a target electrical signal may have a frequency based, at least in part, on a frequency of a first electrical signal and a frequency of a second electrical signal. Of course, such details are merely examples, and claimed subject matter is not so limited.

In an embodiment, skin of a patient may be electrically insulative, while underlying tissue may be conductive. A conductive electrode pad may be used to apply electrical signals to the patient. In particular, it may be desired to transmit such electrical signals through the skin into tissue, such as muscle tissue for example. Insulative skin sandwiched between conductive tissue and a conductive electrode may act as a capacitor. Accordingly, several issues may be considered for transmitting electrical signals through capacitive skin. One issue may be that relatively high frequencies may transmit through a capacitor more so than relatively low frequencies. For example, a 10,000 Hertz (Hz) sinusoidal wave may transmit through a capacitor with less impedance than a 500 Hz sinusoidal wave. Thus, electrical signals having a particular frequency produced by a device may be substantially attenuated inside the patient, under their skin. A TENS (transcutaneous electrical nerve stimulation) unit, for example, may produce a signal having a relatively low frequency, such as about 100 Hz. Thus, such a signal may not be expected to transmit easily though skin or much deeper than just below the skin.

Another issue may be that a waveform of a signal may be transformed as it transmits through a capacitor (e.g., skin or other portion of a patient). Current traveling through a capacitor may be proportional to a time-derivative of the voltage across the capacitor. For example, a 10,000 Hz sine wave entering a capacitor may be transformed to a 10,000 Hz cosine wave upon or after exiting the capacitor. Or, stated another way, the transmitting wave may be phase shifted by 90 degrees. For another example, a square wave entering a capacitor may be transformed to a new waveform having a positive-going pulse (or spike) and a subsequent negative-going pulse for every positive-going square wave. Thus, though a device may produce electrical signals having a particular shape, the signals may have another shape inside the patient, under their skin. Thus, though a TENS unit may generate square wave signals, such particularly shaped signals may not maintain their shape upon or after transmitting through skin.

In some embodiments described herein, devices and techniques are described to account for at least some issues involving transmitting electrical signals through skin of a patient.

In one implementation, a first electrical signal and a second electrical signal may be applied to a patient while the patient is substantially moving (e.g., movement greater than a few millimeters or centimeters). Such movement, which may comprise a component of a patient's therapy, for example, may comprise displacement or rotation of the whole patient, or portions thereof. For example, a human patient may be engaged in repetitive or non-repetitive exercises (e.g., pull-ups, jumps, sit-ups, and so on) or activities (walking, running, swimming, and so on) while electrical energy (e.g., first and second electrical signals) is applied via electrodes to the patient. In another example, because of an inherent nature (e.g., untrained, in a wild setting, non-sedated, and so on) of a non-human patient (e.g., animal, fish, etc.), such a patient may be moving while electrical energy is applied via electrodes.

In another implementation, a first electrical signal and a second electrical signal may be applied to a patient while at least one electrode is submerged in a liquid. For example, electrical energy may be applied to a portion of a patient that is submerged in a water bath. In some implementations of such a situation, at least a portion of a water bath may be considered to comprise an electrode. For example, a foot or hand of a patient may be submerged in a water bath so as to treat the foot or hand, though claimed subject matter is not so limited.

In an embodiment, an apparatus to perform some methods or techniques described herein may comprise an electrical circuit to generate a first electrical signal and a second electrical signal each having a time-varying waveform, wherein a sum of the first electrical signal and the second electrical signal may comprise a target electrical signal having a waveform (e.g., waveshape, frequencies, and so on) to stimulate movement of one or more muscles in a patient. Such a circuit may comprise discrete electronic components and/or a processor, for example. The apparatus may further comprise an output port to provide the first electrical signal and the second electrical signal to the patient via a pair of double-capacitive transcutaneous electrode pads, described below. For example, the output port may be configured to apply a first electrical signal to first electrodes of the pair of double-capacitive transcutaneous electrode pads and to apply a second electrical signal to second electrodes of the pair of double-capacitive transcutaneous electrode pads. In an implementation, each of the double-capacitive transcutaneous electrode pads may comprise a first electrode and a second electrode disposed on a single pad that electrically interacts with dermis and subcutis of skin of a patient. In another implementation, a double-capacitive transcutaneous electrode pad may comprise a system that includes: a first electrode and a second electrode disposed on a single pad; and dermis and subcutis of skin of a patient. Of course, such details are merely examples, and claimed subject matter is not so limited.

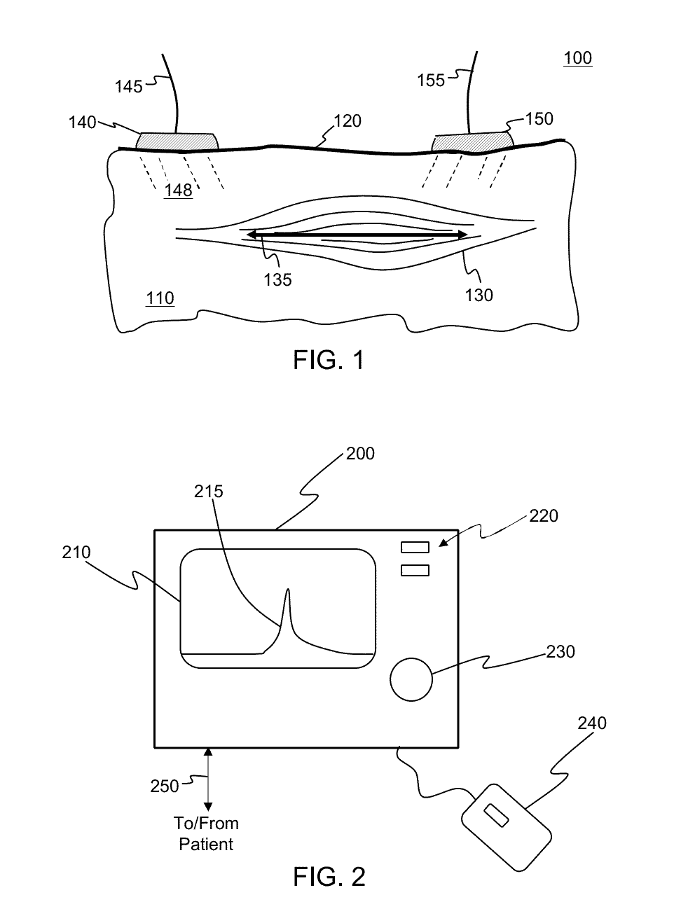

FIG. 1 is a cross-sectional schematic diagram illustrating electrode pads 140 and 150 for applying or distributing one or more electrical signals to a portion 110 of a patient, according to an embodiment 100. Portion 110 may comprise a volume of body mass including, among other things, skin 120 and muscle 130. For sake of clarity, portion 110 may include other biological elements or material that are not shown. For example, such biological elements or material may comprise DNA, normal or cancer cells, fascia, bone, ligaments, organs, plasma, blood vessels, arteries, and so on. Leads 145 and 155 may carry electrical signals to/from electrode pads 140 and 150, respectively. A general flow of electrical signals in the body mass is schematically indicated by dashed lines 148.

Arrow 135 indicates a general direction of motor nerve fibers in muscle 130, for example. Electrical current flowing parallel to motor nerve fibers may stimulate the motor nerve fibers more efficiently compared to the case where current flows perpendicular to the motor nerve fibers.

Electrode pads 140 and 150 may comprise a self-adhesive, metal foil, or conductive rubber (e.g., carbon-impregnated silicone rubber) electrode. In some implementations, a coupling medium may be used to provide a conductive bridge between an electrode pad and skin, such as by filling in voids or gaps, or by increasing conductivity of skin or electrode pad surfaces. A coupling medium may be an integral part of self-adhesive electrode pads, for example. With conductive rubber electrode pads an adhesive gel pad may be used. A coupling gel-pad, which may be solid but soft and flexible, may be both electrically conductive and adhesive. In any case, electrode pads need not be adhesive. Electrode pads may also be strapped onto skin, with or without a coupling medium, or held in place manually by a hand of a therapist or patient, for example. A coupling medium for metal foil electrode pads may comprise an electrode gel or a wetted pad of lint, cotton gauze, or some form of sponge material that absorbs and retains water, for example. Metal electrode pads using spread-able gel or wetted pads may be held in contact to skin by straps or bandages.

An electrode pair used to apply a signal to a patient may comprise a first electrode and a second electrode. The first electrode may comprise a “+” electrode and the second electrode may comprise a “−” electrode, though the symbols “+” and “−” need not indicate positive or negative portions of a signal. For example, such electrodes may deliver current comprising a bipolar sinusoidal waveform, which changes polarity many times per second. In such or similar cases, polarity of a “+” electrode and a “−” electrode may vary in time. In one implementation, such symbols may indicate polarity of one electrode relative to the other electrode. In another implementation, such symbols may indicate an anode for a positive electrode and a cathode for a negative electrode.

FIG. 2 is a schematic diagram illustrating a device 200, which may comprise a medical or therapeutic device, according to an embodiment. Device 200 may apply one or more electrical signals to a patient via port 250, according to an embodiment. Device 200 may generate such signals to be applied to a patient via electrode pads, for example, such as 140 and 150. Such signals may comprise waveforms having any of a number of shapes. For example, a waveform may comprise a sinusoid, a square wave, a sawtooth wave, a low-duty-cycle pulse, a microcurrent wave, or an arbitrarily-shaped wave, and so on. A waveform may comprise one waveshape (or other parameters) for one time span, another waveshape (or other parameters) for a subsequent time span, and so on. Variables of waveforms may include time between pulses, pulse duration, duty cycle, shape of pulses, frequency modulations, amplitude modulations, pulse width modulations, ramping, peak on-times, burst frequencies, offset values, magnitudes, decay rates, and so on. In the example shown, waveform 215 may comprise one cycle of a pulse wave. Such waveforms are merely examples, and claimed subject matter is not limited to any particularly-shaped wave or signal. Device 200 may include a screen 210, which may comprise a touchscreen, for example. Device 200 may include a number of switches 220, knobs 230, or keyboard/mouse 240 to allow a user to manipulate the device, input patient information, adjust parameters of a waveform, and so on, for example.

In one implementation, a graphical representation of waveform 215 may be changed or adjusted by a user via touchscreen 210. In another implementation, a graphical representation of waveform 215 may be changed by a user via mouse 240. In yet another implementation, waveform 215 may be changed in response to feedback or other signal provided at port 250. Of course, such details of device 200 are merely examples, and claimed subject matter is not so limited.

Though device 200 is shown in FIG. 2 to have various features or components, a device may comprise any of a number of configurations. For example, in one embodiment, a device may comprise an amplifier that receives (e.g., wired or wirelessly) and amplifies electronic signals representative of a waveform. Such a device need not include a processor, for example. In one embodiment, a device may comprise a smartphone, mobile phone, touch pad, laptop, or other portable (or non-portable) electronic device. In an implementation, an external amplifier may be used with a smartphone (or other portable electronic device), for example, to amplify relatively small voltage or current amplitudes output by the smartphone to higher values sufficient for application to a patient.

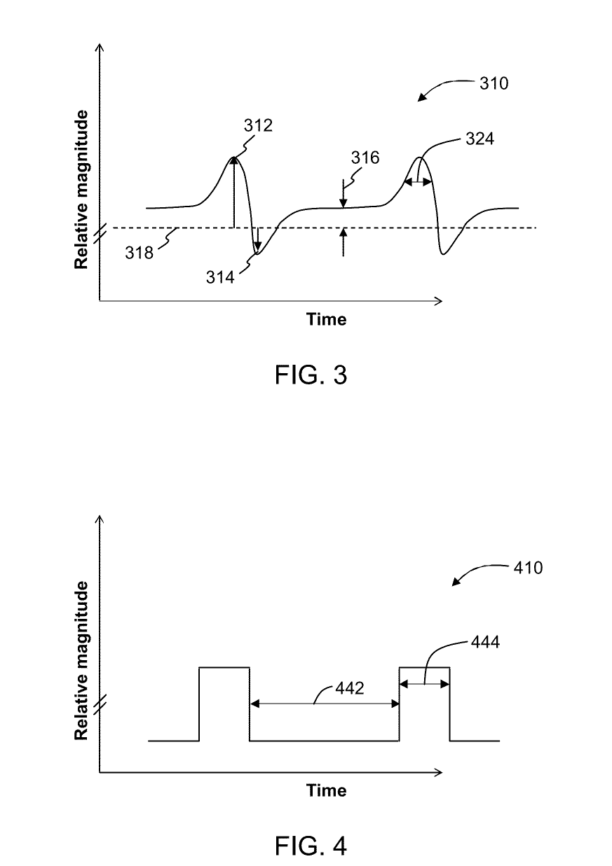

FIGS. 3 and 4 show example signal waves plotted as magnitude of voltage or current versus time, according to embodiments. For example, a first or second signal applied to a patient via electrode pads may comprise any such wave or variation thereof. Of course, there are an endless variety of waves having different shapes or characteristics, and FIGS. 3 and 4 show merely a small number of possibilities. Here, the figures are useful for helping to explain meanings of some terms that are used to describe signal characteristics.

In particular, FIG. 3 shows a wave 310 that includes a positive-going peak having magnitude 312 (e.g., curve is concave downward), a negative-going peak having magnitude 314 (e.g., curve is concave upward), and an offset 316 from a reference level 318, which may be zero volts or ground, for example. Wave 310 also includes a width 324 (e.g., pulse width), which may be described as full width at half max (FWHM). In FIG. 4, wave 410 comprises a square wave having a pulse width 444 and duty cycle that may be described by time 442 between pulses. Of course, any wave may be described by any parameters introduces above, and claimed subject matter is not so limited.

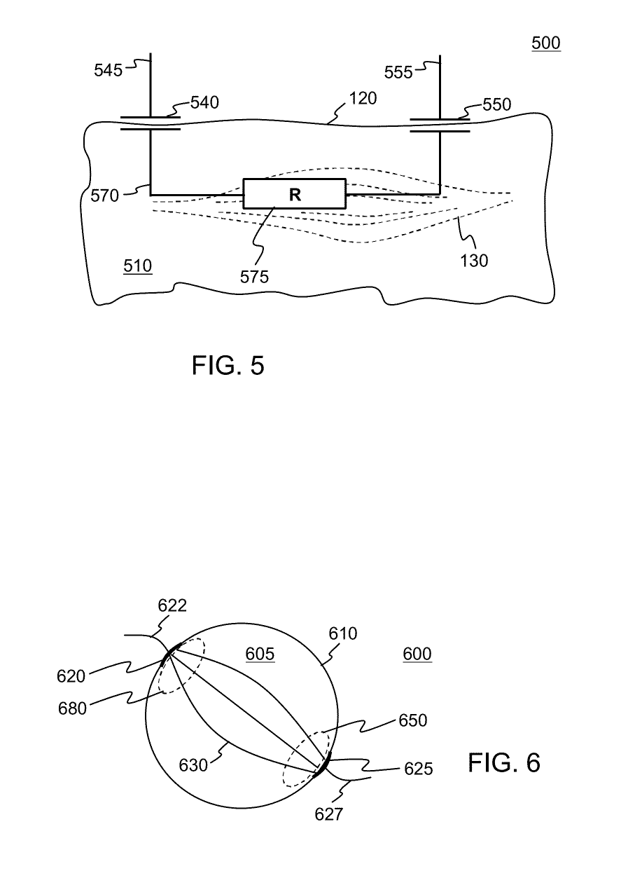

FIG. 5 is a cross-sectional schematic diagram illustrating an electrical component analogue corresponding to the portion 110 of a patient, introduced in FIG. 1, according to an embodiment 500. Skin resistance to an electrical signal may be relatively high depending, at least in part, on frequency of the electrical signal. Thus, electrical signals arriving at skin 120 via leads 545 and 555 may face relatively high resistance of skin 120.

Where two low-resistance regions are separated by a high-resistance region, e.g. a near-insulator, a capacitor may be formed and capacitive effects (e.g., phase shift, impedance, and so on) may occur. Thus, where an electrode pad (e.g., 140 and 150) is separated from nerve or muscle in underlying tissue by skin (more specifically, the stratum corneum), there may be a capacitor. Accordingly, electrode pad 140 may be represented by capacitor 540 and electrode pad 150 may be represented by capacitor 550. Capacitors may impede flow of current of an electrical signal, but an extent of such impedance may depend, at least in part, on pulse duration or frequency of the electrical signal. For direct current (e.g., unidirectional current, 0 Hz) or long-duration slowly-varying pulses of current, skin impedance may be relatively high and electrical energy may be (mostly) dissipated in the stratum corneum of the skin. For short bursts of current, capacitive impedance of the stratum corneum may be relatively low and electrical energy may be (mostly) dissipated in underlying tissues. Membranes of cells of various biological elements may also give rise to capacitance (e.g., two low-resistance regions separated by a high-resistance membrane).

Leads 545 and 555 may carry electrical signals from a signal-generating device (e.g., device 200) to electrode pads represented by capacitors 540 and 550, respectively. Upon or after reaching below the skin, such electrical signals may follow any of a number of paths in and around muscle tissue, bone, cells, and so on. For example, a path carrying an electrical signal through muscle 130 represented by conductor 570 may have impedance 575, which may depend, at least in part, on frequency and/or waveshape of the electrical signal. In one implementation, an electrical signal having a particular frequency may follow one path and another electrical signal having another particular frequency may follow another path. In another implementation, an electrical signal having a particular waveshape (e.g., sinusoid, sawtooth, triangular, square, pulse width, duty cycle, rise/fall time, slope, and so on) may follow one path and another electrical signal having another particular waveshape may follow another path. Thus, as mentioned above, impedance of a path followed by an electrical signal may depend, at least in part, on frequency and/or waveshape of the electrical signal.

An amount of current flowing through tissue may depend, at least in part, on applied voltage between electrode pads 140 and 150 and impedance, according to Ohm's Law, V=IZ, for example. Here, V is applied voltage, Z is impedance, and I is current flow. Current of an electrical signal may flow from one electrode to an opposite electrode along any of a number of paths in a patient. Such paths may depend, at least in part, on electrical and/or chemical properties of internal portions of a patient, such as plasma, muscle, organs, cell structure, just to name a few examples. As mentioned above, a path traveled by an electrical signal may depend, at least in part, on the waveshape of the signal and/or the frequency of the signal, among other things. Though not shown, paths may include any of a number or variety of biological elements (e.g., bone, organs, plasma, cells, tumors, various types of biological tissue, and so on), in addition to muscle tissue 130 shown in FIG. 5.

For example, returning to FIG. 1, current of an electrical signal may flow from electrode pad 140, through skin 120, into underlying tissues (e.g., 130), and then through another layer of skin to electrode pad 150. A total impedance of such a path may comprise a sum of the impedances in each part of the current pathway if the parts are in series (e.g., meaning that current may flow through each part in turn). In an implementation, impedance of electrodes may be relatively low. Moreover, impedance of subcutaneous tissue, which may be highly hydrated, may also be relatively low. Skin impedance, however, may be much higher due, at least in part, to a relatively high impedance of the stratum corneum of skin.

In more detail, skin may comprise the dermis and epidermis. The epidermis may be punctured by various skin appendages, such as sweat gland ducts and hair follicles. Beneath skin is the subcutis, also referred to as superficial fascia or simply subcutaneous tissue. In most areas of a patient, the subcutis may be predominantly adipose (fat storing) tissue. Blood vessels, lymph vessels and nerves may infiltrate the subcutis and dermis, but not the epidermis. The dermis and subcutis have relatively low electrical resistance. The subcutis, which may be adipose tissue, may have relatively low resistance, even though fat is an insulator. The low resistance may be due, in part, to conductive channels (blood and lymph vessels) that may infiltrate the subcutis tissue. Blood or lymph vessels may not infiltrate the epidermis of skin, which may be avascular, meaning that these cells (e.g., keratinocytes) derive their nutrients by diffusion from capillaries in underlying dermis. A basal layer of the epidermis of skin may be metabolically very active, with the cells regularly undergoing mitosis. Keratinocytes, formed and pushed upwards from this layer, may synthesize keratin, which may be retained within the individual cells. In their life cycle, the keratinocytes may move toward the skin surface, becoming less metabolically active as diffusion limits the rate of nutrient supply. Near the surface the cells may die and shrivel, finally forming a scaly shell called the stratum corneum. The stratum corneum may thus comprise a layer of shriveled, dead, dehydrated keratinocytes, which may further comprise packages of keratin. This structure may contribute significantly to the relatively high resistance of skin.

As mentioned above, an electrical signal may follow a path depending, at least in part, on electrical and/or chemical properties of internal portions of a patient. For example, electrical conductivity of muscle may be different from that of bone or a particular organ. Moreover, as an example, electrical conductivity of muscle tissue or bone may depend, at least in part, on the health or density of the muscle tissue or bone (or portion thereof). In the case of muscle tissue, for example, measurements of electrical conductivity of muscle tissue may be used to determine muscle loss or gain in patients with Lou Gehrig's Disease, also known as amyotrophic lateral sclerosis, or ALS. This disease may attack motor neurons that control voluntary muscle movement, leading to muscle weakness and atrophy. As ALS spreads, motor neurons may die off, causing muscles to atrophy. Deteriorating muscles may behave differently from healthy ones, resisting electrical current more, for example. Such variations in behavior may be correlated with disease progression and length of survival of a patient. As another example, electrical conductivity of internal portions of a patient may depend, at least in part, on tissue density, presence of cancer cells, and so on.

Also mentioned above, a path traveled by an electrical signal may depend, at least in part, on the voltage and/or the frequency of a signal applied to a patient via electrodes. For example, a relatively low frequency signal, such as below 10,000 Hz may travel through connective biological tissue, but not through individual cells. As the frequency increases above 10,000 Hz, a signal may begin to penetrate outside layers of cells. Above 100,000 Hz, cell penetration may be substantial. In another example, organ tissue density may vary from organ to organ. As tissue density increases, so does electrical resistance to relatively low frequency signals (e.g., below 10,000 Hz). Accordingly, an electrical signal having one frequency may follow a path different from a path followed by an electrical signal having another frequency.

In examples above, the term “resistance” is used. However, as noted earlier, “impedance” may further describe the case of an electrical signal traveling through internal portions of a patient, particularly if such an electrical signal includes a non-zero frequency or phase. Different internal portions of a patient may have different resistivities and/or different capacitances. Examples above touched on ideas that different biological elements may have different resistivities, which may affect current or voltage of a signal. Moreover, different biological elements may have different capacitances, which may affect current, voltage, or phase of a signal. For example, a time-varying (e.g., a sinusoid) electrical signal may experience a shift in phase between current and voltage based, at least in part, on integrity of muscle tissue (e.g., tissue density, mass, and so on). A phase shift brought about by an electrical signal traveling a path through particular biological tissue may correspond to a capacitive (or inductive) component of impedance of the biological tissue, for example. In a case where impedance is frequency-dependant, an electrical signal having one frequency (or one set of frequencies) may follow a path through biological tissue different from a path followed by an electrical signal having another frequency (or another set of frequencies).

Biological elements may respond to different signals in different ways. For example, a pulse of a signal may activate an action potential of nerve fibers in muscle tissue if a slope of the pulse is sufficiently steep. On the other hand, if a pulse is not steep enough, then the same nerve fibers may accommodate (e.g., “adjust”) to current flow of the pulse so that no action potential is activated. This illustrates an example where applied signals may affect biological elements for which the signals are used to diagnose. For another such example, a 10,000 Hz sinusoidal signal applied to muscle tissue may increase permeability of the muscle tissue. Accordingly, application of particular signals may affect muscle tissue so that resistance of the muscle tissue changes in response to the applied signals. Different applied signals (e.g., different by frequency, waveshape, voltage level, and so on) may affect particular biological elements differently. Thus, for example, different applied signals may give rise to different resistances of a particular biological element.

FIG. 6 is a schematic diagram illustrating electrical stimulation in a portion 605 of a patient, according to an embodiment 600. For example, an electrical signal may be applied to portion 605 via a pair of electrode pads 620 and 625. Portion 605 may comprise any of number of types of biological elements, such as muscle, bone, plasma, and so on. Portion 605 may be surrounded by skin 610, on which are placed electrode pads 620 and 625. For one illustrative example, electrode pad 620 may be placed on a portion of a patient's upper back and electrode pad 625 may be placed on a portion of the patient's lower back. In such a case, portion 605 may represent at least the patient's back below the skin. For another illustrative example, electrode pad 620 may be placed on a portion of a patient's lower right leg and electrode pad 625 may be placed on a portion of the patient's right arm. In such a case, portion 605 may represent at least a large part of the patient's body below the skin.

A lead 622 may comprise a cable or wire to conduct an electrical signal from a device that generates the electrical signal to electrode pad 620. Similarly, a lead 627 may comprise a cable or wire to conduct an electrical signal to electrode pad 625. Leads 622 and 627 may both connect into a port (e.g., 250) of a device such as 200, for example. Electrode pad 620 may comprise a positive electrode and electrode pad 625 may comprise a corresponding negative electrode. Lines 630 represent paths traveled by electrical signals applied to skin 610 via electrode pads 620 and 625. Though not shown, for example, such paths may extend through or across muscle, bone, or other biological elements of portion 605. Dashed ellipse 680 indicates an approximate region of portion 605 where substantial effects of electrical signals applied by electrode pad 620 may occur. Similarly, dashed ellipse 650 indicates an approximate region of portion 605 where substantial effects of electrical signals applied by electrode pad 625 may occur. For example, efficiency of electrical signals for stimulating motor nerves of muscles may depend, at least in part, on the current density of the electrical signals. Current density may decrease as distances from the electrode pads increases due, at least in part, to current spreading. For example, outside regions such as 650 and 680, efficiency of electrical signals for stimulating motor nerves of muscles may be relatively low, while efficiency of the electrical signals within the regions may be relatively high. Unfortunately, a relatively large portion of tissue between 650 and 680 (e.g., along lines 630) may be in a region that experiences relatively weak electrical signals. In embodiments discussed below, double-capacitive transcutaneous electrode pads may be used to enable electrical signals to reach relatively large portions of underlying (e.g., deep) tissue.

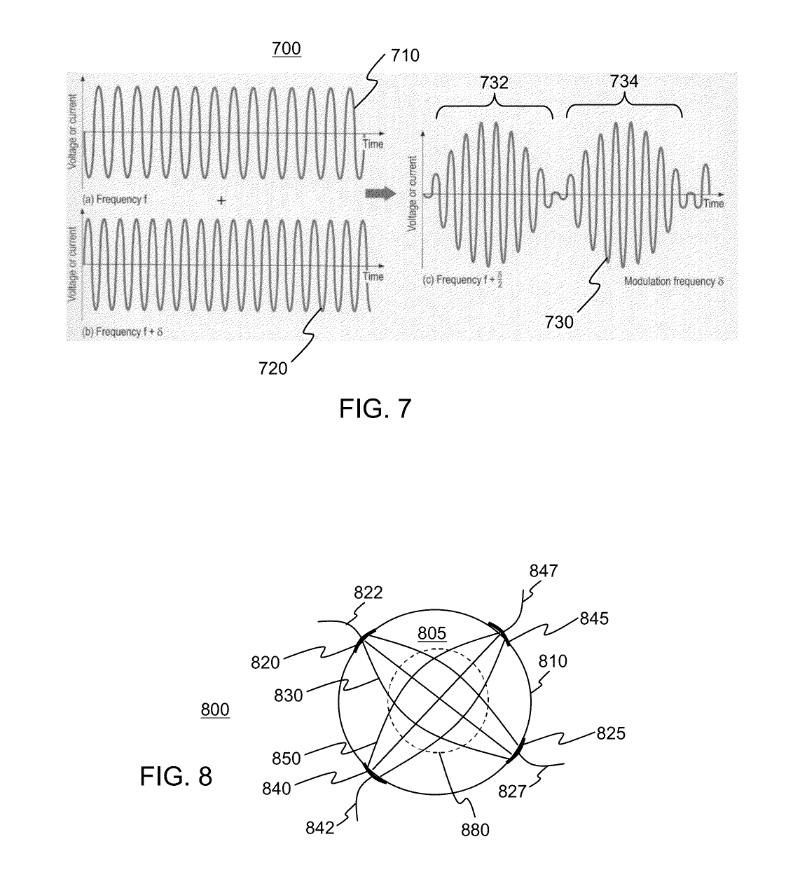

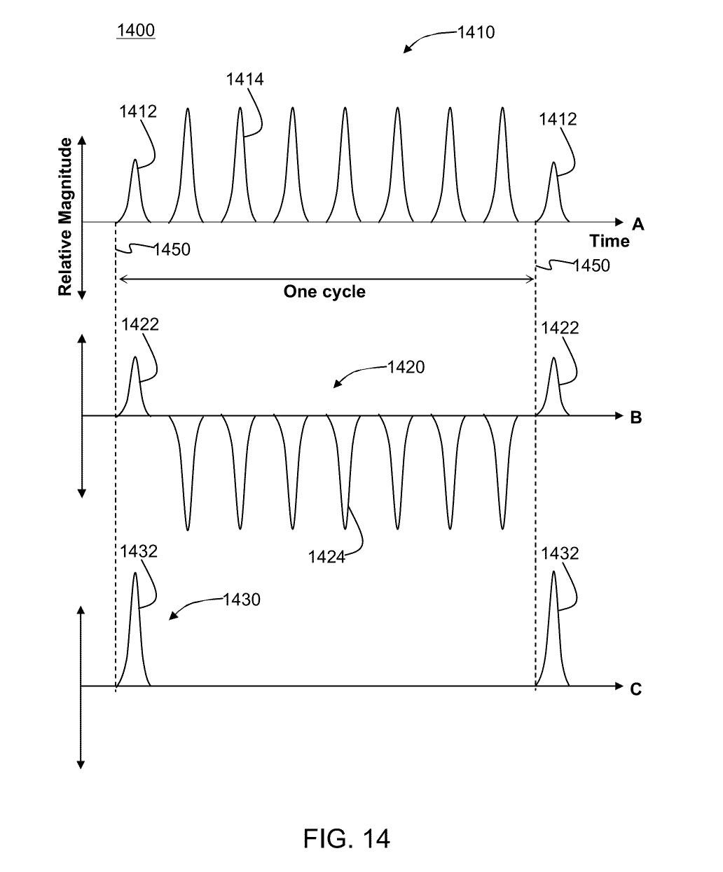

FIG. 7 shows a superposition 730 of two sinusoidal waveforms 710 and 720 having dissimilar frequencies, according to an embodiment 700. For example, if waveform 710 has a frequency of f and waveform 720 has a frequency of f+δ, then superposition 730 may modulate with a beat frequency of δ. If two signals intersect within tissue they ‘interfere’ or superimpose. For example, the total electrical current at any point may comprise the sum of the currents of the two signals. In a particular example, if first signal 710 has a frequency of 4000 Hz, and a frequency of second signal 720 is 4050 Hz, then the resulting superposition signal 730 may have a frequency of modulation of 50 Hz, in this example. This frequency is called the beat frequency: it is equal to the difference in frequency between the two signals. Within tissue the superposition signal may be in the form of sinusoidal bursts 732 and 734 of current with a burst (or beat) frequency of 50 Hz. Frequency of modulation within individual bursts may be referred to as the carrier frequency. FIG. 700 involves first and second signals that are both sinusoids. However, embodiments described below (e.g., FIGS. 14-19) involve first and second signals that may be non-sinusoidal and configured to superpose to a particular target signal.

There are various types of apparatuses for applying electrical energy to a patient. For example, an interference-type apparatus may stimulate structures located within a patient's body, such as muscles or nerves that control muscle action, which may be reached with relatively high frequency signals (e.g., several thousand to tens of thousands of Hertz), but may be mostly responsive to relatively low frequency signals (e.g., tens or hundreds of Hertz). This apparatus may operate by applying two primary signals (e.g., first and second signals 710 and 720) of relatively high, but slightly different, frequencies to a patient's body. The primary signals, due to their relatively high frequency, may penetrate the patient's body and reach the aforementioned structures where they combine and produce a beat signal (e.g., superposition signal 730) having a relatively low frequency that is equal to the slight difference in the frequencies of the primary signals. FIG. 8 depicts such a situation, for example.

FIG. 8 is a schematic diagram illustrating interferential electrical stimulation in a portion 805 of a patient, according to an embodiment 800. In contrast to an embodiment depicted in FIG. 6, which involves one pair of electrode pads for applying a signal to a patient, interferential electrical stimulation may involve two pairs of electrode pads. For example, a first electrical signal may be applied to portion 805 via a first pair of electrode pads 820 and 825. A second electrical signal may be applied to portion 805 via a second pair of electrode pads 840 and 845. First and second electrical signals may comprise signals similar to 710 and 720, respectively, shown in FIG. 7, for example. Portion 805 may comprise any of number of types of biological elements, such as muscle, bone, plasma, and so on. Portion 805 may be surrounded by skin 810, on which are placed the electrode pad pairs. For one illustrative example, first electrode pad pair 820/825 may be placed on a portion of a patient's upper back and second electrode pad pair 840/845 may be placed on a portion of the patient's lower back. In such a case, portion 805 may represent at least the patient's back below the skin.

Leads 822, 827, 842, and 847 may comprise a cable or wire to conduct electrical signals to electrode pads 820, 825, 840, and 845, respectively. Leads 822, 827, 842, and 847 may connect into a port (e.g., 250) of a device such as 200, for example. Lines 830 represent paths traveled by electrical signals applied to skin 810 via electrode pad pairs 820 and 825. Lines 850 represent paths traveled by electrical signals applied to skin 810 via electrode pad pairs 840 and 845. Though not shown, for example, such paths may extend through or across muscle, bone, or other biological elements of portion 805. Dashed circle 880 indicates an approximate region of portion 805 where substantial effects of electrical signals applied by electrode pads 820, 825, 840, and 845 may occur. In particular, circle 880 indicates a region where first and second signals may interfere with one another. Thus, if first and second signals comprise signals 710 and 720, respectively, then their superposition 730 may occur in a region indicated by circle 880, for example. Such a superposition of electrical signals may comprise interferential currents or interferential signals.

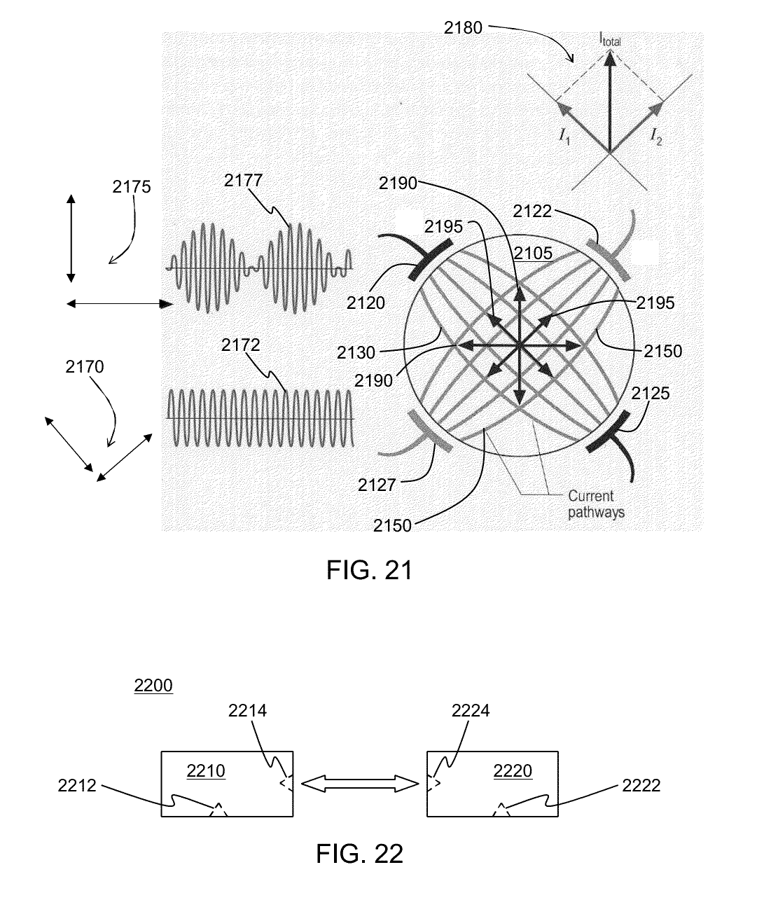

In an embodiment, interferential currents may be relatively highly efficient at stimulation of tissue. For example, in the region of intersection of first and second signals (e.g., where superposition occurs), the reinforcement of the signals may provide a greater total stimulus intensity so maximum stimulation is produced at depth rather than superficially as may occur with stimulation using a single signal and a single pair of electrode pads. Undesirably, however, relatively large portions of 805, such as outside circle 880, may experience relatively weak (e.g., non-superposed signals). Moreover, such signals outside circle 880 may not include a beat frequency, so that frequencies of these signals may be mostly too high to stimulate muscles along 830 or 850. Also, as explained in further detail below (e.g., for FIG. 21), it may be desirable for vectors of superposition signals to be substantially parallel to muscle nerve fibers. Unfortunately, it may be difficult or impossible to place electrode pads 820, 825, 840, and 845 at a combination of positions on skin 810 so that a superposition of signals they provide run substantially parallel with particular muscle(s) that are to be stimulated, for example. In embodiments discussed below, double-capacitive transcutaneous electrode pads may provide a therapist an improved ability (e.g., compared to using other electrode pads) to form electrical signals that run substantially parallel with muscle(s). Of course, such details regarding FIG. 8 are merely examples, and claimed subject matter is not so limited.

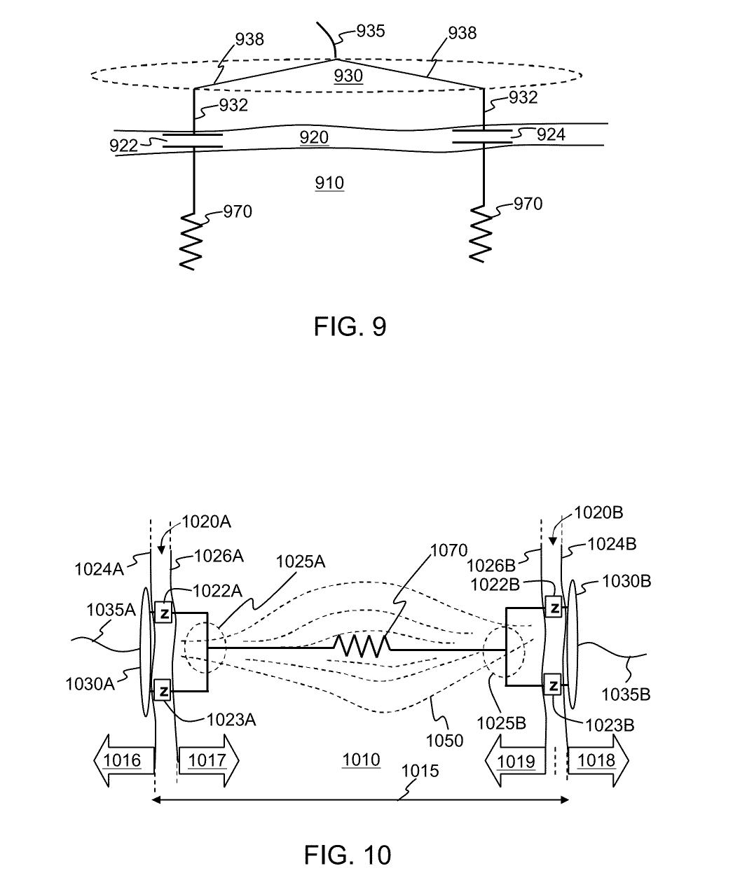

FIG. 9 is a cross-sectional schematic diagram illustrating an electrical component analogue corresponding to a double-capacitive transcutaneous (DCT) electrode pad 930 and a portion of a patient including skin 920 and underlying tissue 910, according to an embodiment. For example, DCT electrode pad 930 may be affixed to skin 920 with an adhesive, elastic wrapping, or merely held in place manually. Alternatively, DCT electrode pad 930 may be contacting skin 920 while pad 930 is slid across skin 920 from location to location on a patient's body. For example, during a therapeutic process, pad 930 may be slid from a patient's lower back region to their upper back region while maintaining substantial contact between pad 930 and skin 920. Here, substantial contact includes situations where at least portions of the pad may momentarily lift away from the skin while sliding across the skin, or applied pressure between the pad and skin may vary.

As discussed in detail below, a DCT electrode may comprise an electrode pad that includes two individual electrodes that are insulated from each other. A single cable 935 may individually connect the two individual electrodes to respective output ports of a device, such as 200, shown in FIG. 2. Accordingly, such a single cable may include two conductors each comprising stranded or a single wire. In other implementations, however, cable 935 need not comprise a single cable. Lines 938 represent conductors that extend from where cable 935 terminates at or near a surface of DCT electrode pad 930 to the individual electrodes, respectively. Lines 932 represent conductivity between the individual electrodes and skin 920, respectively.

As discussed above, skin may behave as an electrical insulator while underlying tissue may behave as a conductor. Accordingly, because an electrode pad is conductive, the skin may be modeled as a capacitor. In FIG. 9, capacitor symbol 922 represents skin capacitance between one of the electrodes of electrode pad 930 and underlying tissue 910. Similarly, capacitor symbol 924 represents skin capacitance between the other one of the electrodes of electrode pad 930 and underlying tissue 910. Resistor symbols 970 represent electrical resistance of underlying tissue 910. One of the electrodes may provide a first signal to skin 920 while the other electrode may provide a second signal to skin 920, for example.

FIG. 10 is a cross-sectional schematic diagram illustrating an electrical component analogue corresponding to a portion of a patient and electrically contacting DCT electrode pads, according to an embodiment. DCT electrode pads 1030A and 1030B may be used to transcutaneously apply electrical energy to a patient. For example, transcutaneously applying electrical energy to a patient may electrically stimulate one or more muscles in underlying tissue 1010 in the patient. DCT electrode pads 1030A and 1030B may be located at any of a number of locations on a patient. For example, DCT electrode pad 1030A may be located on a forearm and 1030B may be located on a hip. In another example, DCT electrode pad 1030A may be temporarily adhered to an upper back region and 1030B may be slid across a region of the lower back while an electrical signal is applied to the patient. DCT electrode pad 1030A may comprise a positive DCT electrode pad and DCT electrode pad 1030B may comprise a corresponding negative DCT electrode pad, described in detail below.

Skin 1020A and skin 1020B may comprise portions or regions of a patient's skin that are just below, or electrically contacting, DCT electrode pads 1030A and 1030B, respectively. Arrow 1015 schematically represents a distance traveled by one or more electrical signals through the patient and between DCT electrode pads 1030A and 1030B. (However, electrical signals need not travel a shortest line between two points, for example. Such signals may spread to some extent in opposite directions to a principle flow of current from one electrode to another, for example.)

Signals may comprise a first electrical signal and a second electrical signal having time-varying waveforms. The sum of the first electrical signal and the second electrical signal may comprise a target electrical signal having a waveform to generate or stimulate substantial motor movement of one or more muscles 1050 in a patient, for example, though claimed subject matter is not limited to a target electrical signal that generates muscle movement. A first electrical signal may be applied to a patient via a first electrode and a second electrical signal may be applied via a second electrode. The first electrode and the second electrode may be disposed on a single DCT electrode pad, 1030A or 1030B. These pads may be contacting skin 1020A and 1020B of the patient so that the first electrode and the second electrode are electrically separated by capacitance of the skin based, at least in part, on dermis and subcutis of the skin. For example, placing such a single electrode pad on skin of a patient may allow the first electrical signal and the second electrical signal to combine below the skin in a region 1025A or 1025B to form the target electrical signal. In one implementation, a target electrical signal may have a frequency based, at least in part, on a frequency of a first electrical signal and a frequency of a second electrical signal. For example, the frequency of a target signal may comprise a beat frequency of a superposition of first and second signals.

In further detail, a first DCT electrode pad 1030A may be electrically contacting skin 1020A on an outer surface 1024A. Block arrow 1016 indicates the region “above” skin 1020A and block arrow 1017 indicates the region “below” skin 1020A. A two-conductor cable 1035A may be electrically attached to DCT electrode pad 1030A. Here, two-conductor cable means a cable with two electrically separated wires, which may comprise single wires or stranded wires insulated from each other, for example. Lamp cord may be one familiar example of a two-conductor cable. Two-conductor cable 1035A, however, may comprise relatively lightweight (e.g., light gauge) medical-grade cable. Tissue 1010, which may be conductive, may comprise any biological elements that are below lower surfaces 1026A and 1026B of skin 1020A and 1020B, respectively. Tissue 1010, for example, may include, among other things, one or more muscles 1050. As discussed above, skin may act as an insulator. Accordingly, skin 1020A sandwiched between DCT electrode pad 1030A and conductive tissue 1010 may behave as a capacitor (in conjunction with DCT electrode pad 1030A and a volume of tissue 1010).

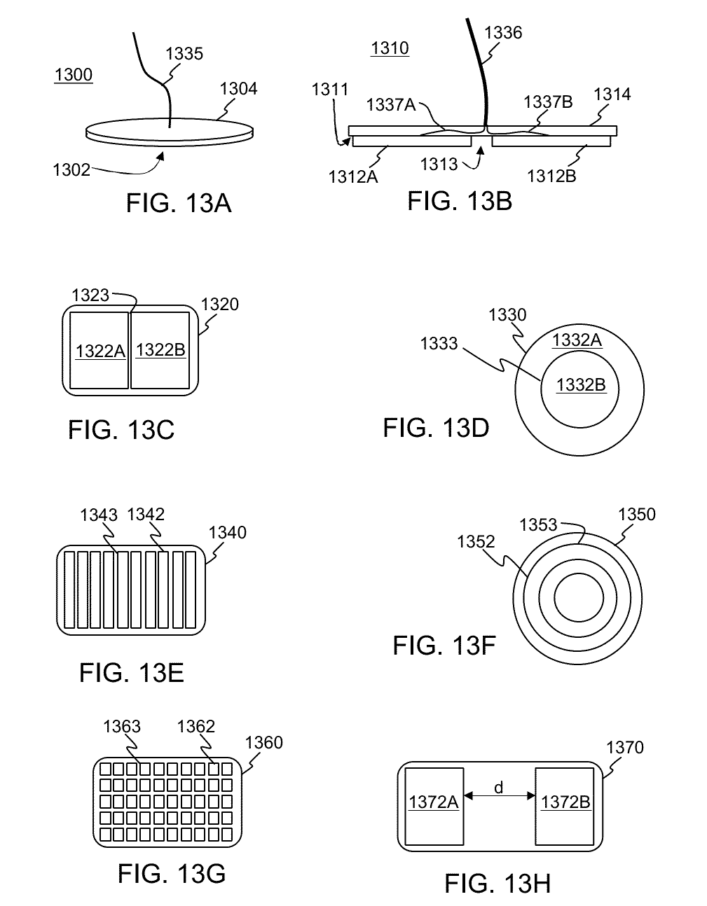

Because DCT electrode pad 1030A may comprise two individual electrodes (e.g., see FIG. 13B), two individual capacitors, 1022A and 1023A, may be formed, in part, by the regions of skin 1020A below the individual electrodes, respectively. Capacitor 1022A may present an impedance Z to a first electrical signal flowing in one electrode and capacitor 1023A may present an impedance Z to a second electrical current flowing in the other electrode. The first and second electrical signals may be modified by impedance Z as the signals travel through skin 1020A. The modified signals may combine (e.g., superpose with each other) in a region 1025A in conductive tissue 1010. Region 1025A may be relatively near lower surface 1026A and just below DCT electrode pad 1030A. For example, region 1025A may extend approximately from lower surface 1026A to a few millimeters or a few centimeters into tissue 1010, though claimed subject matter is not so limited. Accordingly, the superposition of first and second electrical signals may travel through tissue 1010 to region 1025B, just below DCT electrode pad 1030B. Though tissue 1010 may be conductive, it has electrical resistance represented by resistor 1070. For example, inside the human body, resistance between head to toe may be several hundred ohms. As will be discussed in detail below, the superposition of first and second electrical signals may travel through and electrically stimulate one or more muscles 1050, depending, at least in part, on frequency, shape, or intensity of the signal superposition.

A second DCT electrode pad 1030B may be electrically contacting skin 1020B on an outer surface 1024B. Block arrow 1018 indicates the region “above” skin 1020B and block arrow 1019 indicates the region “below” skin 1020B. A two-conductor cable 1035B may be electrically attached to DCT electrode pad 1030B. Here, two-conductor cable means a cable with two electrically separated wires, which may comprise single wires or stranded wires insulated from each other, for example. Two-conductor cable 1035B may comprise relatively lightweight (e.g., light gauge) medical-grade cable, for example. Skin 1020B sandwiched between DCT electrode pad 1030B and conductive tissue 1010 may behave as a capacitor (in conjunction with DCT electrode pad 1030B and a volume of tissue 1010). Because DCT electrode pad 1030B may comprise two individual electrodes (e.g., see FIG. 13B), two individual capacitors, 1022B and 1023B, may be formed, in part, by the regions of skin 1020B below the individual electrodes, respectively. Capacitor 1022B may present an impedance Z to a first electrical signal flowing in one electrode and capacitor 1023B may present an impedance Z to a second electrical current flowing in the other electrode. The first and second electrical signals may be modified by impedance Z as the signals travel through skin 1020B. The modified signals may combine (e.g., superpose with each other) in a region 1025B in conductive tissue 1010. Region 1025B may be relatively near lower surface 1026B and just below DCT electrode pad 1030B. For example, region 1025B may extend approximately from lower surface 1026B to a few millimeters or a few centimeters into tissue 1010, though claimed subject matter is not so limited. Accordingly, the superposition of first and second electrical signals may travel through tissue 1010 to region 1025A, just below DCT electrode pad 1030A. Though tissue 1010 may be conductive, it has electrical resistance represented by resistor 1070. As will be discussed in detail below, the superposition of first and second electrical signals may travel through and electrically stimulate one or more muscles 1050, depending, at least in part, on frequency, shape, and intensity of the signal superposition of signals traveling into the patient via DCT electrode pads 1030A and 1030B.

In the description above for FIG. 8, it was mentioned that it may be difficult or impossible to place electrode pads 820, 825, 840, and 845 at a combination of positions on skin 810 so that a superposition of signals they provide run substantially parallel with particular muscle(s) that are to be stimulated, for example. In contrast, DCT electrode pads 1030A and 1030B may provide a therapist an improved ability (e.g., compared to using other electrode pads) to form electrical signals that run substantially parallel with muscle(s), such as 1050, for example. In an implementation, if muscle 1050 were oriented differently from that shown in FIG. 10, then DCT electrode pads may be relocated so that the signals (or resulting superposition of the signals) they provide are directed substantially parallel to the muscle. In other words, DCT electrode pads may facilitate applying a target signal that runs in tissue 1010 in directions that may be selectable by a therapist by placement the DCT electrode pads at particular locations on a patient. Compare with what may be less desirable scenarios described for embodiment 800, where a therapist may have relatively little control of direction of target signals, for example.

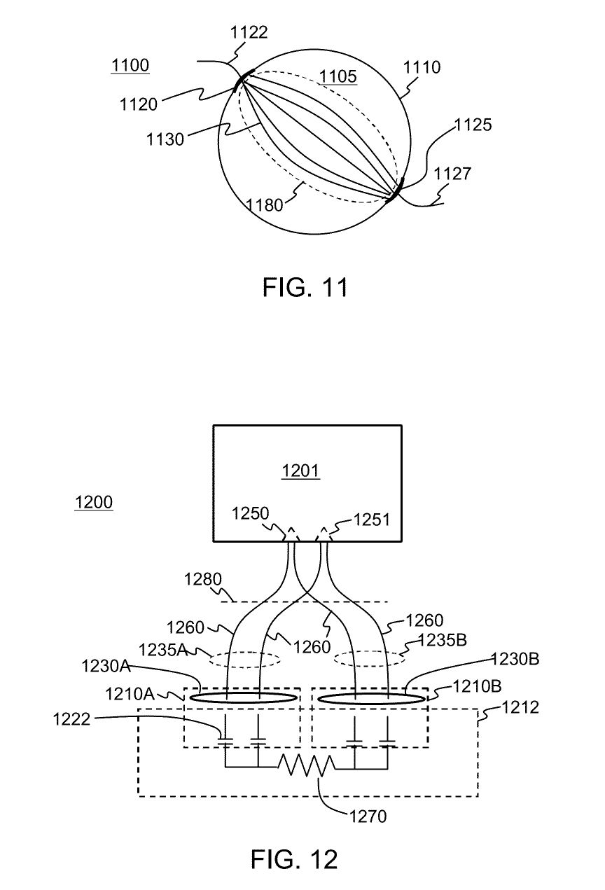

Also, DCT electrodes pads may provide another benefit in that a relatively large portion (e.g., ellipse 1180, shown in FIG. 11) of tissue between electrode pads may experience a target signal. For example, DCT electrodes pads 1030A and 1030B may provide a relatively large portion (e.g., between regions 1025A and 1025B, which may be relatively near the electrode pads) of tissue 1010 between the electrode pads with a target signal. Compare with what may be less desirable scenarios described for embodiments 600 or 800, where relatively less volume of tissue may experience target signals, for example. Of course, such details regarding FIG. 10 are merely examples, and claimed subject matter is not so limited.

FIG. 11 is a schematic diagram illustrating electrical stimulation in a portion 1110 of a patient, according to an embodiment 1100. Embodiments 1100 and 600 (FIG. 6) may be similar to each other in some respects. However, DCT electrode pads are used in embodiment 1100 whereas single-electrode electrode pads are used in embodiment 600. Portion 1110 may be similar to the portion between DCT electrode pads 1030A and 1030B shown in FIG. 10. An electrical signal may be applied to portion 1105 via a pair of DCT electrode pads 1120 and 1125. Portion 1105 may comprise any of number of types of biological elements or tissue, such as muscle, bone, plasma, and so on. Portion 1105 may be surrounded by skin 1110, on which are placed DCT electrode pads 1120 and 1125. For one illustrative example, DCT electrode pad 1120 may be placed on a portion of a patient's upper back and DCT electrode pad 1125 may be placed on a portion of the patient's lower back. In such a case, portion 1105 may represent tissue of at least the patient's back below the skin. For another illustrative example, DCT electrode pad 1120 may be placed on a portion of a patient's lower right leg and DCT electrode pad 1125 may be placed on a portion of the patient's right arm. In such a case, portion 605 may represent tissue of at least a large part of the patient's body below the skin.

A lead 1122 may comprise a cable or wire pair to conduct electrical signals to DCT electrode pad 1120. Similarly, a lead 1127 may comprise a cable or wire pair to conduct electrical signals to DCT electrode pad 1125. In one implementation, leads 1122 and 1127 may both connect into a port (e.g., 250) of a device such as 200. In another implementation, leads 1122 and 1127 may respectively connect into ports 1250 and 1251 of a device 1201, shown in FIG. 12, for example. Lines 1130 represent paths traveled by electrical signals applied to skin 1110 via DCT electrode pads 1120 and 1125. Though not shown, for example, such paths may extend through or across muscle, bone, or other biological elements of portion 1105. Dashed ellipse 1180 indicates an approximate region of portion 1105 where substantial effects of electrical signals applied by DCT electrode pads 1120 and 1125 may occur. For example, outside this region, efficiency of electrical signals for stimulating motor nerves of muscles may be relatively low, while efficiency of the electrical signals within the regions may be relatively high. Compared to sizes of such regions noted in FIGS. 6 and 8, region 1180 is relatively large and extends between DCT electrode pads 1120 and 1125. Accordingly, compared to embodiments shown in FIGS. 6 and 8 that do not involve any DCT electrode pads, effects of electrical signals applied by DCT electrode pads 1120 and 1125 may encompass a relatively large portion of 1105. Of course, such details regarding FIG. 11 are merely examples, and claimed subject matter is not so limited.

FIG. 12 is a schematic diagram illustrating an electronic device 1201 and DCT electrode pads 1230A and 1230B for electrical stimulation, according to an embodiment 1200. In the figure, an electrical analogue to the DCT pads in contact with a portion 1212 of a patient is shown. As discussed above, skin may behave as a capacitor, and tissue below the skin may behave as a resistor. Accordingly, portions of skin under individual electrodes of DCT electrode pads 1230A and 1230B are represented by capacitors 1222 and tissue that carries a superposition target signal is represented by resistor 1270. In one implementation, where DCT electrode pad 1230A is in contact with skin of portion 1212, region 1210A may represent a volume of interaction among the DCT electrode pad, the skin, and the underlying tissue beneath the skin. For example, such interaction may involve frequency-based filtering (e.g., by skin capacitance) of signals provided by cable 1235A and/or superposing the signals below the skin (e.g., via conductance of the underlying tissue). Similarly, where DCT electrode pad 1230B is in contact with skin of portion 1212, region 1210B may represent a volume of interaction among the DCT electrode pad, the skin, and the underlying tissue beneath the skin. For example, such interaction may involve frequency-based filtering of signals provided by cable 1235B and/or superposing the signals below the skin.

Cables 1235A and 1235B to each carry two individual signals may include two individual conductors 1260 (e.g., single wire or stranded wires) insulated between each other. Device 1201 may include a number of ports for electrical signals to be applied to a patient via DCT electrode pads. Several configurations may be implemented. In one implementation, a port 1250 may provide positive polarity signals to both DCT electrode pads 1230A and 1230B, while port 1251 may provide negative polarity signals to both DCT electrode pads 1230A and 1230B. Here, each DCT electrode pad may include a positive electrode and a negative electrode. In another implementation, however, wires carrying particular polarities may be “crossed” inside device 1201 so that a first port may provide both positive and negative polarity signals to DCT electrode pad 1230A and a second port may provide both positive and negative polarity signals to DCT electrode pad 1230B. Dashed line 1280 schematically represents where such ports may be located (e.g., in lieu of ports 1250 and 1251) in the latter implementation.

FIG. 13A is a perspective view of a DCT electrode pad 1304, according to an embodiment 1300. Though not visible in this figure, DCT electrode pad 1304 may include two individual electrodes on face 1302, which may be intended to contact skin of a patient. Cable 1335 may comprise two individual wires (e.g., single wire or stranded wire) to respectively supply electrical signals to the two individual electrodes. Though cable 1335 is shown to be attached at a center top region of pad 1304, cable 1335 may instead attach anywhere on the top region or the circumferential edge (or perimeter edge in the case of a noncircular pad), for example.

DCT electrode pad 1304 may comprise an insulative substrate on which may be located individual conductive electrodes, which may be insulated from each other. Insulation material may comprise rubber, silicone-type materials, plastic-type materials, and so on. Pad 1304 may be flexible or pliable so as to conform to a surface shape of a portion of a patient, for example. Electrode material may comprise a conductive metal-infused rubber or plastic-type compound. Each individual electrode may have an area slightly less than half the surface area of pad 1304, for example. Though DCT electrode pad of embodiment 1300 may be circular, a DCT electrode pad may have any shape, such as square, triangular, oval, and so on. Also, in other embodiments, a DCT electrode pad may include more than two electrodes. Accordingly, a cable (e.g., 1335) may include more than two conductors (e.g., one conductor per one electrode). Claimed subject matter is not limited to any particular DCT electrode pad configuration.

FIG. 13B is a cross-sectional view of a DCT electrode pad 1314, according to an embodiment 1310. DCT electrode pad 1314 may include two individual electrodes 1312A and 1312B on face 1311. The two electrodes may be intended to contact skin of a patient. Cable 1336 may comprise two individual wires (e.g., single wire or stranded wire) to respectively supply electrical signals to the individual electrodes 1312A and 1312B via conductors 1337A and 1337B, respectively. Though cable 1336 is shown to be attached at a center top region of pad 1314, cable 1336 may instead attach anywhere on the top region or the perimeter edge, for example.

DCT electrode pad 1314 may comprise an insulative substrate on which may be located individual conductive electrodes 1312A and 1312B, which may be insulated from each other. In one implementation, conductive electrodes 1312A and 1312B may comprise portions of the insulative substrate that may be modified to be conductive (e.g., a doping-type process). Insulation material may comprise rubber, silicone-type materials, plastic-type materials, and so on. Pad 1314 may be flexible or pliable so as to conform to a surface shape of a portion of a patient, for example. Electrode material may comprise a conductive metal-infused rubber or plastic-type compound. Each individual electrode may have an area slightly less than half the surface area of pad 1314, for example, though claimed subject matter is not so limited. A gap 1313 may be between electrodes 1312A and 1312B to maintain an insulative barrier between the two electrodes. Sizes may be exaggerated in FIG. 13B. For example, gap 1313 may be anywhere in a range from about a micron to a few millimeters, though claimed subject matter is not limited to any particular size. Thicknesses of electrodes 1312A and 1312B may also be anywhere in a range from about a micron to a few millimeters. A sponge-type electrode may be up to a centimeter thick, for example.

In one implementation, gap 1313 may comprise a recessed region that may not contact skin while electrodes 1312A and 1312B are in contact with the skin. Such non-contact with skin may be desirable in that the skin may maintain a relatively cool temperature compared to skin that is in contact with an overlaying material. A recessed region may allow air to circulate or the skin to “breathe” so as to help avoid sweating or other moisture-increasing action (e.g., from sweat glands in the skin). Such increased skin moisture may increase conductivity of the surface of the skin, which may undesirably allow “crosstalk” between electrodes 1312A and 1312B. Without such moisture, for example, signals from electrodes 1312A and 1312B find less skin resistance “vertically” through the relatively thin skin to the underlying tissue as opposed to skin resistance “horizontally” from one electrode to the other electrode. In other embodiments of DCT electrode pads, such as those shown in FIGS. 13C-13H, a recessed region configured to not contact skin while adjacent electrodes are in contact with the skin may be between any adjacent electrodes, for example. A conductive gel may be used between electrodes and skin, as mentioned above.

DCT electrode pad of embodiment 1310 may have any shape, such as circular, square, triangular, oval, and so on. Also, in other embodiments, a DCT electrode pad may include more than two electrodes. Accordingly, a cable (e.g., 1336) may include more than two conductors (e.g., one conductor per one electrode). Claimed subject matter is not limited to any particular DCT electrode pad configuration.

FIGS. 13C-13H are bottom views of example embodiments of various configurations of double or multi-capacitive transcutaneous electrode pads. DCT electrode pads of these example embodiments may include more than two electrodes (e.g., multi-capacitive transcutaneous electrode pads). Claimed subject matter is not limited to any particular DCT electrode pad configuration.

FIG. 13C shows a bottom view of a DCT electrode pad 1320 that includes electrodes 1322A and 1322B, which need not have areas the same as each other. These electrodes may have any shape and/or proportion of area of pad 1320. In one implementation, DCT electrode pad 1320 may be the same as 1314, so that FIG. 13C is a bottom view of embodiment 1310. Though pad 1320 and electrodes 1322A and 1322B are shown to be rectangular, pad 1320 and electrodes 1322A and 1322B may have any of a number of shapes, such as elliptical, oval, or circular, just to name a few examples. In some implementations, DCT electrode pad 1320 may include a recessed region 1323 configured to not contact skin while electrodes 1322A and 1322B are in contact with the skin. Such a recessed region may be several millimeters wide and may be recessed by several millimeters, for example.

FIG. 13D shows a bottom view of a DCT electrode pad 1330 that includes electrodes 1332A and 1332B, which need not have areas the same as each other. These electrodes may have any shape and/or proportion of area of pad 1330. The circular line between electrodes 1332A and 1332B indicates an insulative region that electrically separates electrodes 1332A and 1332B, for example. Though pad 1330 and electrodes 1332A and 1332B are shown to be circular, pad 1330 and electrodes 1332A and 1332B may have any of a number of shapes, such as elliptical, oval, or rectangular, just to name a few examples. In some implementations, DCT electrode pad 1330 may include a recessed region 1333 configured to not contact skin while electrodes 1332A and 1332B are in contact with the skin.

FIG. 13E shows a bottom view of a multi-capacitive transcutaneous (MCT) electrode pad 1340 that includes a plurality of electrodes 1342, which need not have areas the same as each other. These electrodes may have any shape and/or proportion of area of pad 1340. The area between electrodes 1342 indicates an insulative region that electrically separates electrodes 1342 from one another, for example. Though pad 1340 and electrodes 1342 are shown to be rectangular, pad 1340 and electrodes 1342 may have any of a number of shapes, such as elliptical, oval, or circular, just to name a few examples. In some implementations, DCT electrode pad 1340 may include recessed regions 1343 configured to not contact skin while electrodes 1342 are in contact with the skin. Such recessed regions may be several millimeters wide and may be recessed by several millimeters, for example.

FIG. 13F shows a bottom view of an MCT electrode pad 1350 that includes a plurality of electrodes 1352, which need not have areas the same as each other. These electrodes may have any shape and/or proportion of area of pad 1350. The circular line between adjacent electrodes 1352 indicates an insulative region that electrically separates electrodes 1352 from one another, for example. Though pad 1350 and electrodes 1352 are shown to be circular, pad 1350 and electrodes 1352 may have any of a number of shapes, such as elliptical, oval, or rectangular, just to name a few examples. In some implementations, DCT electrode pad 1350 may include recessed regions 1353 configured to not contact skin while electrodes 1352 are in contact with the skin. Such recessed regions may be several millimeters wide and may be recessed by several millimeters, for example.

FIG. 13G shows a bottom view of an MCT electrode pad 1360 that includes a plurality of electrodes 1362, which need not have areas the same as each other. These electrodes may have any shape and/or proportion of area of pad 1360. The area between electrodes 1362 indicates an insulative region that electrically separates electrodes 1362 from one another, for example. Though pad 1360 and electrodes 1362 are shown to be rectangular, pad 1360 and electrodes 1362 may have any of a number of shapes, such as elliptical, oval, or circular, just to name a few examples. In some implementations, DCT electrode pad 1360 may include recessed regions 1363 configured to not contact skin while electrodes 1362 are in contact with the skin. Such recessed regions may be several millimeters wide and may be recessed by several millimeters, for example.

FIG. 13H shows a bottom view of a DCT electrode pad 1370 that includes electrodes 1372A and 1372B, which need not have areas the same as each other. These electrodes may have any shape and/or proportion of area of pad 1370. In one implementation, DCT electrode pad 1370 may be the same as 1314, so that FIG. 13C is a bottom view of embodiment 1310. Though pad 1370 and electrodes 1372A and 1372B are shown to be rectangular, pad 1370 and electrodes 1372A and 1372B may have any of a number of shapes, such as elliptical, oval, or circular, just to name a few examples. “d” indicates that an insulative region of any length or area may be present between or among individual electrodes (including the pluralities of electrodes shown in the previous figures, for example.

FIGS. 14-19 show examples of first and second signals that may be provided to skin of a patient via first and second electrodes of a DCT electrode pad. Though claimed subject matter is not so limited, first and second signals may be designed so that their superposition, which may occur in tissue under skin, comprises a target signal having desirable characteristics for therapeutically treating a patient. Individually, sans superposition, such first or second signals may not have such desirable characteristics, but first and second signal may have other useful characteristics, such as for efficiently transmitting through a patient's skin, maintaining a relatively low voltage at a electrode-skin interface, and/or reducing discomfort to the patient, just to name a few examples.

For a particular example, first and second signals may have frequencies that are relatively high, which may allow the signals to efficiently transmit through a patient's skin and/or reduce discomfort to the patient, who may not be able to feel high frequency signals. However, such high frequencies may be unable to efficiently stimulate particular physical processes in a patient, such as muscle movement, cellular processes, break up scar tissue, improve blood circulation, and so on. But, a superposition (e.g., target signal) of these high frequency signals may have a relatively low frequency and may be able to stimulate such particular physical processes, for example. Though two signals and two electrodes in a DCT electrode pad are discussed, similar techniques may involve more than two signals and more than two electrodes, and claimed subject matter is not so limited.

FIG. 14 is a plot of characteristics for first and second electrical signals and their superposition as a function of time, according to an embodiment 1400. Time axis A is used to plot first electrical signal 1410, time axis B is used to plot second electrical signal 1420, and time axis C is used to plot a superposition 1430 of the first and second electrical signals. For example, first electrical signal 1410 may be applied to skin of a patient via a first electrode (e.g., 1312A) of a DCT electrode pad (e.g., 1314) and second electrical signal 1420 may be applied to the skin of the patient via a second electrode (e.g., 1312B) of the DCT electrode pad. Embodiment 1400 demonstrates that a target signal (e.g., 1430) having a relatively low frequency may be produced by a superposition of two signals (e.g., 1410 and 1420) having relatively high frequencies. This may be beneficial, for example, where relatively high frequency signals pass through skin with less impedance compared to the case for relatively low frequency signals. However, a relatively low frequency signal may be desirable to act upon motor neurons of muscle tissue for muscle stimulation, for example. Thus, a DCT electrode pad may be used to apply relatively high frequency first and second signals through skin, wherein the first and second signals produce a relatively low frequency target signal upon or after traveling through the skin.

For illustrative purposes, one cycle plus an extra pulse of the subsequent cycle of the electrical signals are plotted. One cycle is shown to span between times indicated by dashed lines 1450. For sake of simplicity in the examples shown in FIG. 14, zero magnitude (vertical direction) of each plot may be considered to coincide with the time axis of the plot. For example, pulse 1422 may be positive and pulses 1424 may be negative. However, any of the waveforms or signals shown herein, unless specified otherwise, may be vertically offset by any amount. In other words, zero magnitude in the vertical direction may be shifted upward or downward by any amount. For example, signal 1420 may be offset so that upper portions of pulses 1424 are positive, and merely bottom portions of pulses 1424 are negative. In some implementations, it may be desirable to have a substantially net-zero current applied to a patient to avoid skin burns, for example. In other words, an area between a curve and a zero-axis, or a time-integral of a signal (e.g., 1410, 1420, 1430, and so on), may desirably be substantially zero. Accordingly, example signal plots shown herein (e.g., including those of FIGS. 14-19) may be offset by any amount to achieve desirable therapy for a patient. Any of the signals may also be inverted. Claimed subject matter is not limited in this respect.

First electrical signal 1410 may comprise positive-going pulses with one of the pulses 1412 having a magnitude about half that of the remaining individual pulses 1414. Here, the one pulse that is different from the remaining pulses may be called a “feature pulse” and the remaining pulses may be called “carrier pulses”. There may be more than one feature pulse per cycle. Though a Gaussian peak is shown, feature and carrier pulses may comprise any of a number of shapes, such as square, ramp, triangle, and double exponential, just to name a few examples. Though pulse widths of the feature pulse and carrier pulse are shown to be the same, this need not be the case: A feature pulse may have a greater or lesser pulse width than that of the carrier pulses.

First electrical signal 1410 may have any frequency. For example, frequencies of 1410 may be in a range from about 1,000 Hz to 20,000 Hz, though claimed subject matter is not so limited. In some embodiments, frequencies may extend into the megahertz range. In an implementation, frequencies may be selected so that electrical signals may pass through a patient's capacitive skin with relatively low impedance. Accordingly, first and second electrical signals having frequencies greater than about several kilohertz may be desirable. In the case of first electrical signal 1410, the carrier pulse frequency may be about seven times greater than the frequency of the feature pulse, since there are about seven carrier pulses for every one feature pulse per cycle, in this particular example. Of course, however, this is merely an example, and claimed subject matter is not limited by such a frequency relationship.

Second electrical signal 1420 may comprise mostly negative-going pulses with one of the pulses 1422 being positive-going and having about half the magnitude of the remaining individual pulses 1424. Second electrical signal 1420 may be called a “bipolar” signal because the signal is both positive and negative at times during a cycle. In contrast, first electrical signal 1410 may be called “unipolar” signal because the signal is only positive (or it may be only negative and be called a unipolar signal, for example) during a cycle. As above, the one pulse that is different from the remaining pulses may be called a “feature pulse” and the remaining pulses may be called “carrier pulses”. There may be more than one feature pulse per cycle. Though a Gaussian peak is shown, feature and carrier pulses may comprise any of a number of shapes, such as square, ramp, triangle, and double exponential, just to name a few examples. Though pulse widths of the feature pulse and carrier pulse are shown to be the same, this need not be the case: A feature pulse may have a greater or lesser pulse width than that of the carrier pulses.

Second electrical signal 1420 may have any frequency. For example, a frequency of 1420 may similar to that of first electrical signal 1410, though claimed subject matter is not so limited. In an implementation, the frequencies of first and second electrical signals may be substantially equal so that a superposition 1430 of first and second electrical signals may comprise a target waveform having a desired shape and/or frequencies, for example. Herein, unless otherwise specified, the plurality “frequencies” may imply that a signal may comprise multiple Fourier frequency components depending, at least in part, on the shape of the waveform of the signal, for example.

Superposition 1430 may comprise a pulse 1432, which is a sum of feature pulses 1412 and 1422. Carrier pulses 1414 and 1424 sum to substantially zero, or “cancel out”.

Accordingly, superposition 1430, comprising a target signal, may have a frequency that is substantially less than that of the first and second signals 1410 and 1420.

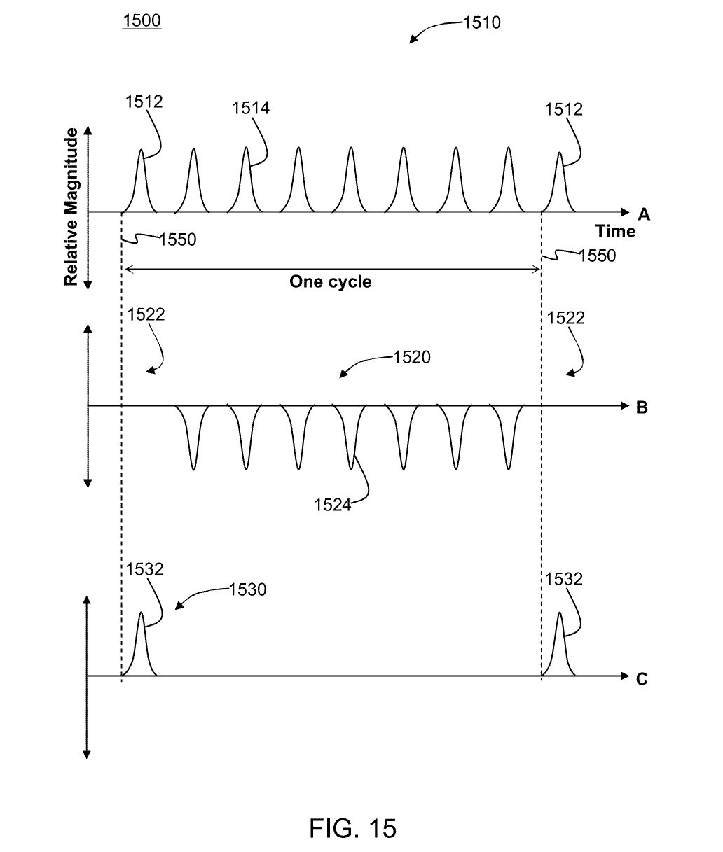

FIG. 15 is a plot of characteristics for first and second electrical signals and their superposition as a function of time, according to an embodiment 1500. Time axis A is used to plot first electrical signal 1510, time axis B is used to plot second electrical signal 1520, and time axis C is used to plot a superposition 1530 of the first and second electrical signals. Embodiment 1500 may be similar to embodiment 1400, though embodiment 1500 may only involve unipolar signals, for example.

First electrical signal 1510 may be applied to skin of a patient via a first electrode (e.g., 1312A) of a DCT electrode pad (e.g., 1314) and second electrical signal 1520 may be applied to the skin of the patient via a second electrode (e.g., 1312B) of the DCT electrode pad. Embodiment 1500 demonstrates that a target signal (e.g., 1530) having a relatively low frequency may be produced by a superposition of two signals (e.g., 1510 and 1520) having relatively high frequencies. As explained above, this may be beneficial, for example, where relatively high frequency signals pass through skin with less impedance compared to the case for relatively low frequency signals. However, a relatively low frequency signal may be desirable to act upon motor neurons of muscle tissue for muscle stimulation, for example. Thus, a DCT electrode pad may be used to apply relatively high frequency first and second signals through skin, wherein the first and second signals produce a relatively low frequency target signal upon or after traveling through the skin.

For illustrative purposes, one cycle plus an extra pulse of the subsequent cycle of the electrical signals are plotted. One cycle is shown to span between times indicated by dashed lines 1550. For sake of simplicity in the examples shown in FIG. 15, zero magnitude (vertical direction) of each plot may be considered to coincide with the time axis of the plot. For example, pulses 1512 and 1514 are positive and pulses 1524 are negative. However, any of the waveforms or signals shown herein, unless specified otherwise, may be vertically offset by any amount. In other words, zero magnitude in the vertical direction may be shifted upward or downward by any amount. For example, signal 1520 may be offset so that upper portions of pulses 1524 are positive and bottom portions of pulses 1524 are negative. Any of the signals may also be inverted. Claimed subject matter is not limited in this respect.

First electrical signal 1510 may comprise unipolar positive-going pulses with one of the pulses 1512 having about the same magnitude as the remaining individual pulses 1514. As mentioned above, the one pulse that is different from the remaining pulses may be called a “feature pulse” and the remaining pulses may be called “carrier pulses”. There may be more than one feature pulse per cycle. Though a Gaussian peak is shown, feature and carrier pulses may comprise any of a number of shapes, such as square, ramp, triangle, and double exponential, just to name a few examples. Though pulse widths of the feature pulse and carrier pulse are shown to be the same, this need not be the case: A feature pulse may have a greater or lesser pulse width than that of the carrier pulses.

First electrical signal 1510 may have any frequency. For example, frequencies of 1510 may be in a range from about 1,000 Hz to 20,000 Hz, though claimed subject matter is not so limited. In some embodiments, frequencies may extend into the megahertz range. In an implementation, frequencies may be selected so that electrical signals may pass through a patient's capacitive skin with relatively low impedance. Accordingly, first and second electrical signals having frequencies greater than about several kilohertz may be desirable. In the case of first electrical signal 1510, the carrier pulse frequency may be about seven times greater than the frequency of the feature pulse, since there are about seven carrier pulses for every one feature pulse per cycle, in this particular example. However, in the particular case of 1510, feature pulse and carrier pulse may be similar, so that frequencies of feature pulses and carrier pulses may not be different. Also, such a frequency relationship between first and second signals is merely an example, and claimed subject matter is not so limited.

Second electrical signal 1520 may comprise unipolar negative-going pulses with the feature pulse 1522 comprising a “null” pulse in phase with the feature pulse 1512 of the first electrical signal 1510. As explained above, though a Gaussian peak is shown, feature and carrier pulses may comprise any of a number of shapes, such as square, ramp, triangle, and double exponential, just to name a few examples. Though pulse widths of the feature pulse and carrier pulse are shown to be the same, this need not be the case: A feature pulse may have a greater or lesser pulse width than that of the carrier pulses.

Second electrical signal 1520 may have any frequency. For example, a frequency of 1520 may similar to that of first electrical signal 1510, though claimed subject matter is not so limited. In an implementation, the frequencies of first and second electrical signals may be substantially equal so that a superposition 1530 of first and second electrical signals may comprise a target waveform having a desired shape and/or frequencies, for example.

Superposition 1530 may comprise a pulse 1532, which is a sum of feature pulse 1512 and null pulse 1522. Carrier pulses 1514 and 1524 sum to substantially zero, or “cancel out”. Accordingly, superposition 1530, comprising a target signal, may have a frequency that is substantially less than that of the first and second signals 1510 and 1520. For example, target signal 1530 may have a frequency that efficiently stimulates muscles, whereas signals 1510 and 1520 may have frequencies that are too high to stimulate the muscles.

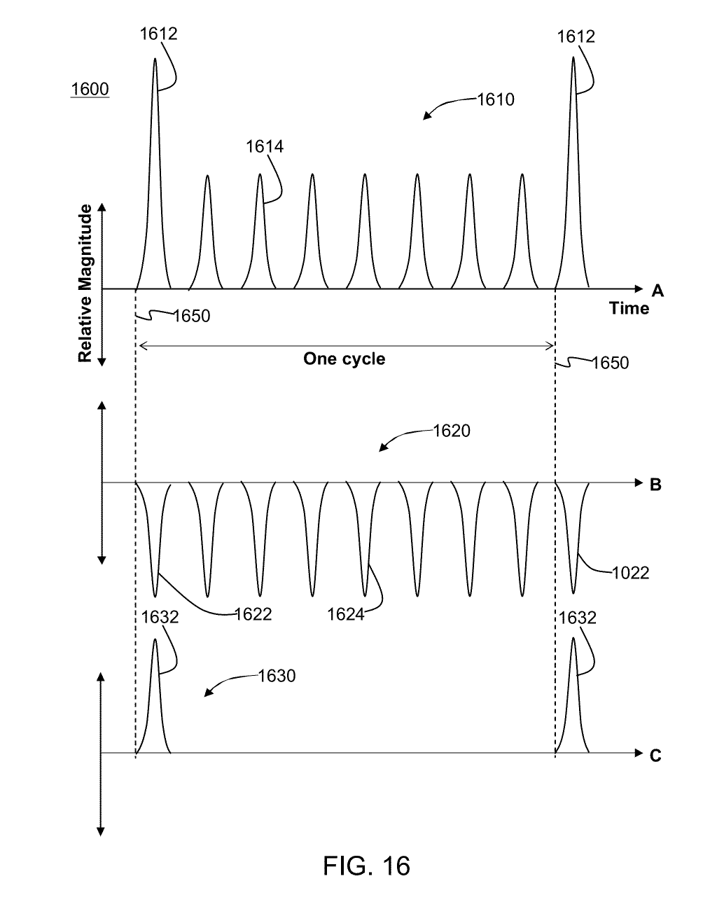

FIG. 16 is a plot of characteristics for first and second electrical signals and their superposition as a function of time, according to an embodiment 1600. Time axis A is used to plot first electrical signal 1610, time axis B is used to plot second electrical signal 1620, and time axis C is used to plot a superposition 1630 of the first and second electrical signals. For example, first electrical signal 1610 may be applied to skin of a patient via a first electrode (e.g., 1312A) of a DCT electrode pad (e.g., 1314) and second electrical signal 1620 may be applied to the skin of the patient via a second electrode (e.g., 1312B) of the DCT electrode pad. Embodiment 1600 demonstrates that a target signal (e.g., 1630) having a relatively low frequency may be produced by a superposition of two signals (e.g., 1610 and 1620) having relatively high frequencies. Thus, a DCT electrode pad may be used to apply relatively high frequency first and second signals through skin, wherein the first and second signals produce a relatively low frequency target signal upon or after traveling through the skin.

For illustrative purposes, one cycle plus an extra pulse of the subsequent cycle of the electrical signals are plotted. One cycle is shown to span between times indicated by dashed lines 1650. For sake of simplicity in the examples shown in FIG. 16, zero magnitude (vertical direction) of each plot may be considered to coincide with the time axis of the plot. For example, pulses 1612 and 1614 are positive and pulses 1622 and 1624 are negative. However, any of the waveforms or signals shown herein, unless specified otherwise, may be vertically offset by any amount. In other words, zero magnitude in the vertical direction may be shifted upward or downward by any amount. For example, signal 1610 may be offset (e.g., moved upward) so that lower portions of pulses 1614 are negative, and upper portions of pulses 1624 are positive. Also, any of the signals may also be inverted. Claimed subject matter is not limited in this respect.

First electrical signal 1610 may comprise unipolar positive-going pulses with one of the pulses 1612 having a magnitude about twice that of the remaining individual pulses 1614. As explained above, the one pulse that is different from the remaining pulses may be called a “feature pulse” and the remaining pulses may be called “carrier pulses”.

There may be more than one feature pulse per cycle. Though a Gaussian peak is shown, feature and carrier pulses may comprise any of a number of shapes, such as square, ramp, triangle, and double exponential, just to name a few examples. Though pulse widths of the feature pulse and carrier pulse are shown to be the same, this need not be the case: A feature pulse may have a greater or lesser pulse width than that of the carrier pulses.

First electrical signal 1610 may have any frequency. For example, frequencies of 1610 may be in a range from about 1,000 Hz to 20,000 Hz, though claimed subject matter is not so limited. In some embodiments, frequencies may extend into the megahertz range. In the case of first electrical signal 1610, the carrier pulse frequency may be about seven times greater than the frequency of the feature pulse, since there are about seven carrier pulses for every one feature pulse per cycle, in this particular example. Of course, however, this is merely an example, and claimed subject matter is not limited by such a frequency relationship.

Second electrical signal 1620 may comprise unipolar negative-going pulses comprising feature pulse 1622 and carrier pulses 1624. Of course, there may be more than one feature pulse per cycle, and claimed subject matter is not limited in this respect. Though a Gaussian peak is shown, feature and carrier pulses may comprise any of a number of shapes, such as square, ramp, triangle, and double exponential, just to name a few examples. Though pulse widths of the feature pulse and carrier pulse are shown to be the same, this need not be the case: A feature pulse may have a greater or lesser pulse width than that of the carrier pulses.

Second electrical signal 1620 may have any frequency. For example, a frequency of 1620 may similar to that of first electrical signal 1610, though claimed subject matter is not so limited. In an implementation, the frequencies of first and second electrical signals may be substantially equal so that a superposition 1630 of first and second electrical signals may comprise a target waveform having a desired shape and/or frequencies, for example. Herein, unless otherwise specified, the plurality “frequencies” may imply that a signal may comprise multiple Fourier frequency components depending, at least in part, on the shape of the waveform of the signal, for example.

Superposition 1630 may comprise a pulse 1632, which is a sum of feature pulses 1612 and 1622. Carrier pulses 1614 and 1624 sum to substantially zero, or “cancel out”.

Accordingly, superposition 1630, comprising a target signal, may have a frequency that is substantially less than that of the first and second signals 1610 and 1620.

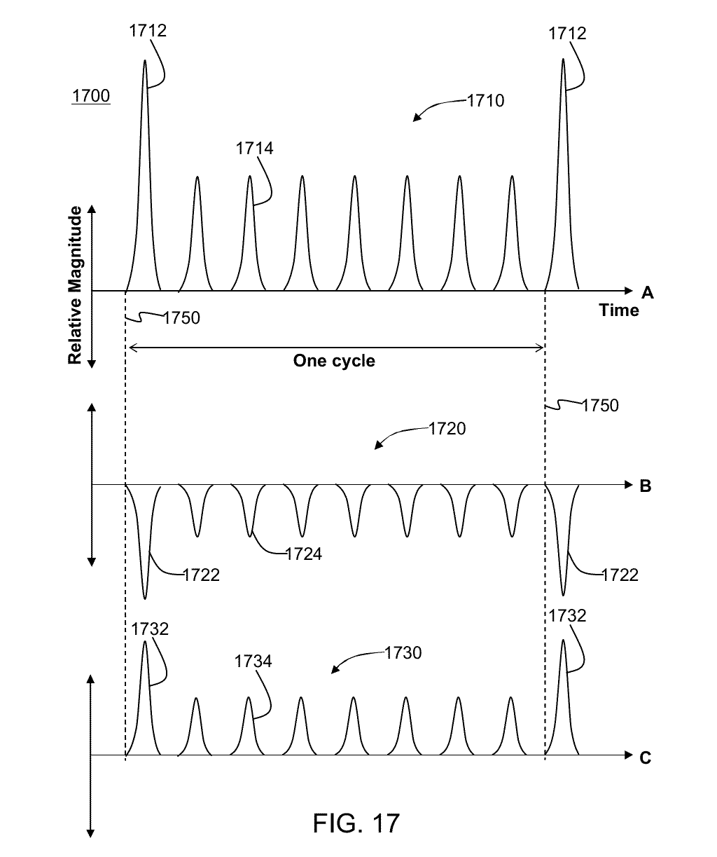

FIG. 17 is a plot of characteristics for first and second electrical signals and their superposition as a function of time, according to an embodiment 1700. For illustrative purposes, one cycle of the electrical signals is plotted, plus an extra pulse of the subsequent cycle. One cycle is shown to span between times indicated by dashed lines 1750. Time axis A is used to plot first electrical signal 1710, time axis B is used to plot second electrical signal 1720, and time axis C is used to plot a superposition 1730 of the first and second electrical signals. Embodiment 1700 may be similar to embodiment 1600, though embodiment 1700 may involve carrier signals that do not “zero-out” or cancel, for example.

In particular, superposition 1730 may comprise a pulse 1732, which is a sum of feature pulses 1712 and 1722. Carrier pulses 1714 and 1724 need not sum to substantially zero, or “cancel out”, as in embodiment 1600. Accordingly, superposition 1730, comprising a target signal, may have a frequency of the feature pulse 1732 that is substantially less than that of the carrier frequencies of first and second signals 1710 and 1720. 1730 may also comprise carrier pulses 1734. Depending, at least in part, on frequencies of signals of embodiment 1700, feature pulses 1732 may stimulate muscle motion in a patient. Though carrier pulses 1734 may have a frequency too high to stimulate the muscles, carrier pulses 1734 may improve blood flow or have a therapeutic effect at a cellular level of the patient, for example.

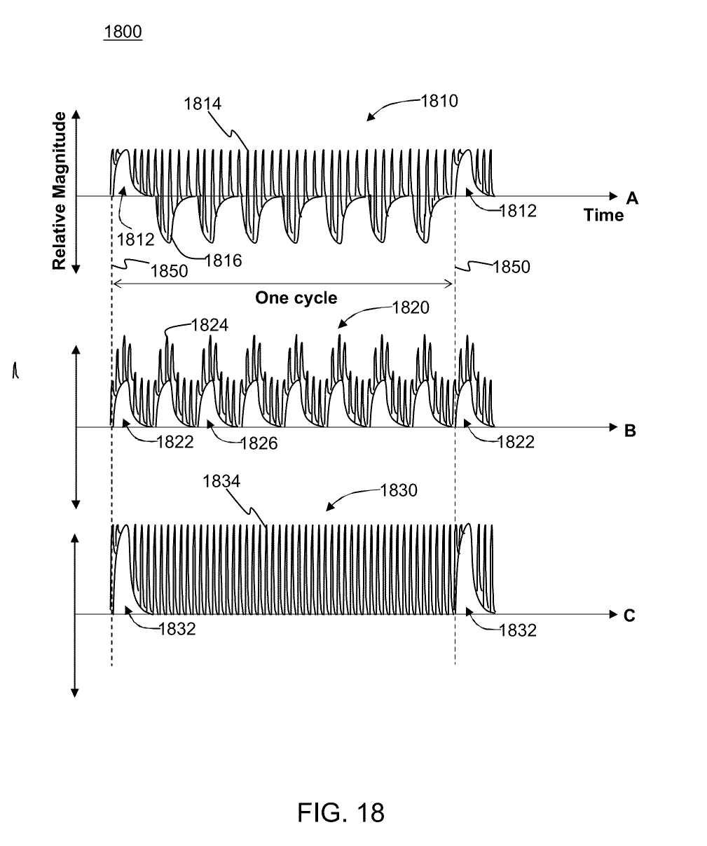

FIG. 18 is a plot of characteristics for first and second electrical signals and their superposition as a function of time, according to an embodiment 1800. Time axis A is used to plot first electrical signal 1810, time axis B is used to plot second electrical signal 1820, and time axis C is used to plot a superposition 1830 of the first and second electrical signals. For example, first electrical signal 1810 may be applied to skin of a patient via a first electrode (e.g., 1312A) of a DCT electrode pad (e.g., 1314) and second electrical signal 1820 may be applied to the skin of the patient via a second electrode (e.g., 1312B) of the DCT electrode pad.

For illustrative purposes, one cycle plus several extra pulses of the subsequent cycle of the electrical signals are plotted. One cycle is shown to span between times indicated by dashed lines 1850. For sake of simplicity in the examples shown in FIG. 18, zero magnitude (vertical direction) of each plot may be considered to coincide with the time axis of the plot. For example, pulses 1812, 1822, and 1824 are positive. Also, signal 1830 is positive. However, any of the waveforms or signals shown herein, unless specified otherwise, may be vertically offset by any amount. In other words, zero magnitude in the vertical direction may be shifted upward or downward by any amount. For example, signals 1810 and/or 1820 may be offset (e.g., moved upward or downward) so that lower portions of superposition signal 1830 are negative, and upper portions are positive. Also, any of the signals may also be inverted. Claimed subject matter is not limited in this respect.

First electrical signal 1810 may comprise bipolar pulses. Pulses 1812 may be considered to be feature pulses. Pulses 1814 may be considered to be first carrier pulses and pulses 1816 may be considered to be second carrier pulses. There may be more than one feature pulse per cycle. Shapes of the feature and carrier pulses may be similar or different. In the example of embodiment 1800, feature pulses 1812 may comprise a double-exponential shape. Portions of signal 1810 may comprise negative-going pulses 1816, which may also comprise a double-exponential shape. However, feature and carrier pulses may comprise any of a number of shapes, such as square, ramp, triangle, double exponential, or any combination thereof, just to name a few examples. Though pulse widths of the feature pulse and carrier pulse may be depicted in the figures as having a particular proportion to one another, this need not be the case.

First electrical signal 1810 may have any set of frequencies. For example, frequencies of features pulses 1812 of signal 1810 may be in a range from about 1.0 Hz to about 1,000 Hz. Frequencies of first or second carrier pulses 1814 or 1816 of signal 1810 may be in a range from about 1,000 Hz to about 20,000 Hz, though claimed subject matter is not limited to any frequency ranges. In some embodiments, frequencies may extend into the megahertz range.

In the case of first electrical signal 1810, the second carrier pulse frequency may be about seven times greater than the frequency of the feature pulse, since there are about seven second carrier pulses for every one feature pulse per cycle, in this particular example. Also, the first carrier pulse frequency may be about forty times greater than the frequency of the feature pulse, since there are about forty first carrier pulses for every one feature pulse per cycle. However, these are merely examples, and claimed subject matter is not limited by such frequency relationships.

Second electrical signal 1820 may comprise unipolar positive-going pulses comprising feature pulse 1822, first carrier pulses 1824, and second carrier pulses 1826. Of course, there may be more than one feature pulse per cycle, and claimed subject matter is not limited in this respect. Second electrical signal 1820 may have any set of frequencies. For example, frequencies of 1820 may similar to that of first electrical signal 1810, though claimed subject matter is not so limited. In an implementation, the frequencies of first and second electrical signals may be substantially equal so that a superposition 1830 of first and second electrical signals may comprise a target waveform having a desired shape and/or frequencies, for example. Again, unless otherwise specified, the plurality “frequencies” may imply that a signal may comprise multiple Fourier frequency components depending, at least in part, on the shape of the waveform of the signal, for example.

Superposition 1830 may comprise a pulse 1832, which is a sum of feature pulses 1812 and 1822. A superposition of carrier pulses 1814 and 1824 may comprise carrier pulses 1834 of target signal 1830. Accordingly, superposition 1830 (e.g., a target signal) may comprise a summed periodic-exponential signal that includes a relatively low frequency feature pulse 1832 (e.g., for stimulating muscles) and a relatively high frequency carrier pulse 1834 (e.g., for stimulation blood circulation or cellular-level responses).

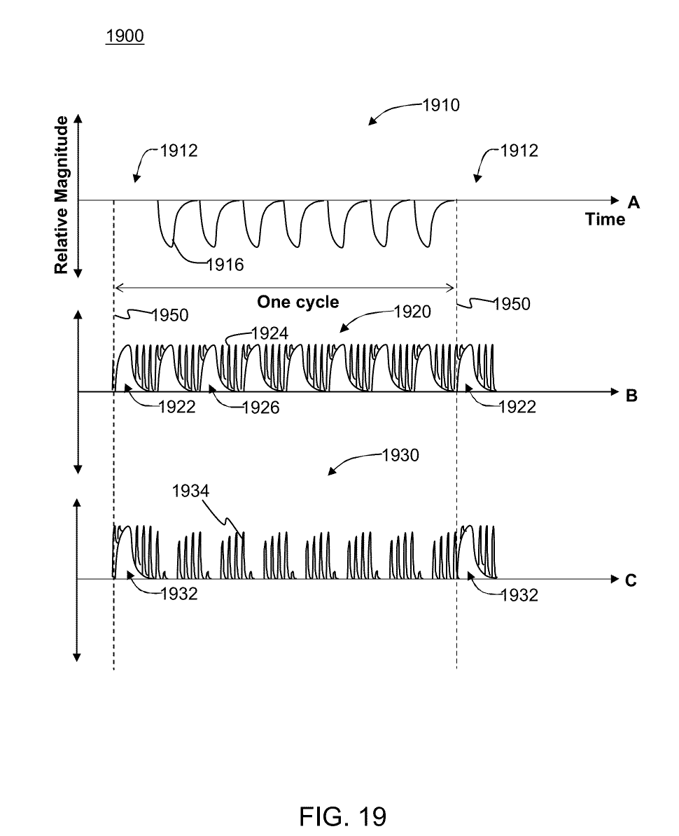

FIG. 19 is a plot of characteristics for first and second electrical signals and their superposition as a function of time, according to an embodiment 1900. Time axis A is used to plot first electrical signal 1910, time axis B is used to plot second electrical signal 1920, and time axis C is used to plot a superposition 1930 of the first and second electrical signals. For example, first electrical signal 1910 may be applied to skin of a patient via a first electrode (e.g., 1312A) of a DCT electrode pad (e.g., 1314) and second electrical signal 1920 may be applied to the skin of the patient via a second electrode (e.g., 1312B) of the DCT electrode pad.

For illustrative purposes, one cycle plus several extra pulses of the subsequent cycle of the electrical signals are plotted. One cycle is shown to span between times indicated by dashed lines 1950. For sake of simplicity in the examples shown in FIG. 19, zero magnitude (vertical direction) of each plot may be considered to coincide with the time axis of the plot. For example, pulses of signals 1920 and 1930 are positive and pulses of signal 1910 are negative. However, any of the waveforms or signals shown herein, unless specified otherwise, may be vertically offset by any amount. In other words, zero magnitude in the vertical direction may be shifted upward or downward by any amount. For example, signals 1910 and/or 1920 may be offset (e.g., moved upward or downward) so that lower portions of superposition signal 1930 are negative, and upper portions are positive. Also, any of the signals may also be inverted. Claimed subject matter is not limited in this respect.

First electrical signal 1910 may comprise unipolar negative-going pulses. Pulses 1912 may comprise “null” pulses and be considered to be feature pulses. Pulses 1916 may be considered to be carrier pulses. In the example of embodiment 1900, carrier pulses 1916 may comprise a double-exponential shape. However, feature and carrier pulses may comprise any of a number of shapes, such as square, ramp, triangle, double exponential, or any combination thereof, just to name a few examples.

First electrical signal 1910 may have any set of frequencies. For example, frequencies of features pulses 1912 of signal 1910 may be in a range from about 1.0 Hz to about 1,000 Hz. Frequencies of carrier pulses 1916 of signal 1910 may be in a range from about 1,000 Hz to about 20,000 Hz, though claimed subject matter is not limited to any frequency ranges. In some embodiments, frequencies may extend into the megahertz range.

In the case of first electrical signal 1910, the carrier pulse frequency may be about seven times greater than the frequency of the feature pulse, since there are about seven second carrier pulses for every one feature pulse per cycle, in this particular example. Of course, however, these are merely examples, and claimed subject matter is not limited by such frequency relationships.

Second electrical signal 1920 may comprise unipolar positive-going pulses comprising feature pulse 1922, first carrier pulses 1924, and second carrier pulses 1926.

Of course, there may be more than one feature pulse per cycle, and claimed subject matter is not limited in this respect. Second electrical signal 1920 may have any set of frequencies. For example, frequencies of 1920 may similar to that of first electrical signal 1910, though claimed subject matter is not so limited. In an implementation, the frequencies of first and second electrical signals may be substantially equal so that a superposition 1930 of first and second electrical signals may comprise a target waveform having a desired shape and/or frequencies, for example.

Superposition 1930 may comprise a pulse 1932, which is a sum of feature pulses 1912 and 1922. A superposition of carrier pulses 1916 and first carrier pulses 1924 may comprise carrier pulses 1934 of target signal 1930. Accordingly, superposition 1930 (e.g., a target signal) may comprise a “triple exponential signal” (or “triple exponential wave”) that includes a relatively low frequency feature pulse 1932 (e.g., for stimulating muscles) and a relatively high frequency carrier pulse 1934 modulating at an intermediate frequency. Such a carrier pulse may stimulate blood circulation or cellular-level responses of a patient, for example.

In one embodiment, an apparatus for providing electrical stimulation to one or more muscles in a patient may comprise means for generating a first time-varying signal and a second time-varying signal. For example, such means may comprise digital or analog (or a combination thereof) electronic circuitry that may include a processor for executing code or one or more function generators (e.g., see FIGS. 22-24), though any of a number of other circuits may be used, and claimed subject matter is not so limited. The apparatus may further comprise means for applying the first time-varying signal and the second time-varying signal to a patient. For example, such means may comprise a DCT electrode pad, though claimed subject matter is not so limited. The apparatus may further comprise means for summing the first time-varying signal and the second time-varying signal below skin of the patient (e.g., via a DCT electrode pad interacting with conductive tissue below skin) at a particular location of the patient to produce a triple exponential signal (e.g., 1930), wherein the triple exponential signal may comprise: a feature pulse (e.g., 1932) for stimulating movement of the one or more muscles and having a frequency below about 1000 Hz; and a carrier pulse (e.g., 1934) having a frequency greater than about 1000 Hz, wherein the carrier pulse is amplitude modulated.

Of course, such details of a triple exponential signal are merely examples, and claimed subject matter is not so limited.

In plots shown in FIGS. 14-19, drawing accuracy (e.g., so that a signal plotted on axis C comprises a superposition of signals plotted on axes A and B) is intended to be sufficient to demonstrate aspects of example embodiments 1400 through 1900. Accordingly, any inaccuracies in the plots (e.g., a signal plotted on axis C may not comprise a precise superposition of signals plotted on axes A and B) are not intended and should not be used to interpret or contradict corresponding text describing the plots. Instead, the plots are used merely to help explain concepts of the embodiments.

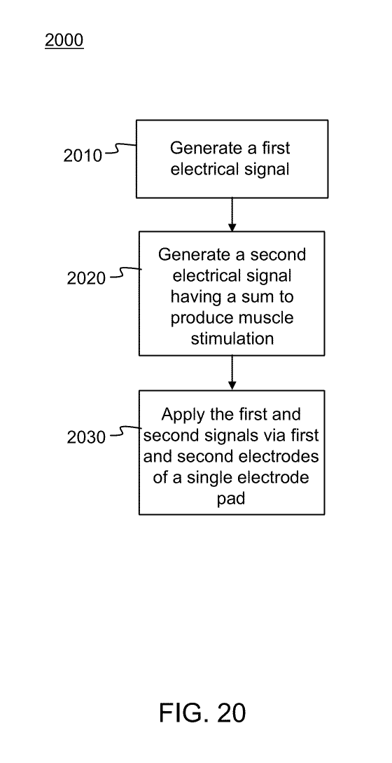

FIG. 20 is a flow diagram of a process 2000 for applying electrical signals to a patient for, among other things, stimulating one or more muscles of the patient, according to an embodiment. For example, process 2000 may comprise transcutaneously applying electrical energy to a patient to electrically stimulate one or more muscles, modify cellular processes, or increase blood flow, just to name a few examples. In an implementation, a system or device, such as those shown in FIG. 2 or 22-24, for example, may perform such a method or technique. At block 2010 a first electrical signal having a time-varying waveform may be generated. At block 2020, a second electrical signal also having a time-varying waveform may be generated. In one implementation, a first electrical signal and a second electrical signal may comprise non-sinusoidal waveforms, such as those shown in FIGS. 14-19, for example. The sum of the first electrical signal and the second electrical signal may comprise a target electrical signal having a waveform to generate or stimulate substantial motor movement of one or more muscles in a patient, for example, though claimed subject matter is not limited to a target electrical signal that generates muscle movement. Signals having frequencies of 600 Hz or less may stimulate motor movement, just to give a numerical example. Claimed subject matter is not so limited.

At block 2030, the first electrical signal may be applied to the patient via a first electrode and the second electrical signal may be applied to the patient via a second electrode at a particular location on the patient. In one example implementation, such a particular location on a patient may be more than about one inch apart from one or more muscles in the patient. In other words, muscles may be stimulated by applying signals via electrodes that are substantially far away from the muscles, such as an inch away or several feet away (e.g., electrodes applied on a lower leg may stimulate muscles in the lower back of a patient). The first electrode and the second electrode may be on a single electrode pad, such as a DCT electrode pad, for example. The single electrode pad may be applied on skin of the patient so that the first electrode and the second electrode are electrically separated by capacitance of the skin based, at least in part, on capacitance presented by dermis and subcutis of the skin. For example, placing such a single electrode pad on skin of a patient may allow the first electrical signal and the second electrical signal to superpose below the skin to form the target electrical signal. In one implementation, a target electrical signal may have a frequency based, at least in part, on a frequency of a first electrical signal and a frequency of a second electrical signal.

In one implementation, a frequency of a first electrical signal and a frequency of a second electrical signal may be greater than about 2000 Hertz and a frequency of a target electrical signal may be less than about 600 Hertz.

In one implementation, a first electrical signal may comprise a first set of pulses that are 180 degrees out of phase with a second set of pulses in a second electrical signal, such as in the case for pulses 1414 and 1424 shown in FIG. 14, for example. In another implementation, a target electrical signal may comprise a summed periodic-exponential signal, such as signal 1830 shown in FIG. 18, for example.

In one implementation, both first and second signals need not be applied to a patient at the same time via a DCT electrode pad. For example, a first signal may be applied for a time on one of the electrodes in a DCT electrode pad, and subsequently a second signal may be introduced to the other electrode of the DCT electrode pad. Slowly increasing amplitude of a signal while maintaining amplitude of another signal may be beneficial in some therapies.

In one implementation, a DCT electrode pad may comprise a positive-polarity DCT electrode pad of a circuit generating first and second electrical signals. In such a case, the first electrical signal may be applied to a patient via a first negative-polarity electrode and the second electrical signal may be applied to the patient via a second negative-polarity electrode to a particular location on the patient. A negative-polarity DCT electrode pad may include the first negative-polarity electrode and the second negative-polarity electrode. In other words, the first and second electrical signal may be applied to the patient via a negative-polarity DCT electrode pad of the circuit generating the signals. For example, a positive-polarity DCT electrode pad may be placed at one location of a patient's skin and a negative-polarity DCT electrode pad may be placed at another location of a patient's skin. In such a case, one or more muscles may be electrically between the respective locations of the positive- and negative-polarity DCT electrode pads.

FIG. 21 shows superpositions of two sinusoidal waveforms having dissimilar frequencies and a schematic diagram illustrating electrical stimulation in a portion 2105 of a patient, according to an embodiment. As explained below, arrows 2190 and 2195 indicate net current flow in different directions for an electrode configuration similar to that shown in FIG. 8, for example. Current vector addition 2180 pictorially indicates how such current vectors may add.

As explained above, if interferential currents intersect in tissue, the currents may interfere (e.g., add) so that a resulting stimulus intensity may be burst modulated, such as that of signal 730 shown in FIG. 7, for example. Because of varying effects of different travel directions of current in tissue, however, this may be somewhat of an oversimplification. For example, if currents of signals 710 and 720 in FIG. 7 ran parallel, and were of equal intensity, then they may interfere so that a resulting stimulus intensity may comprise signal 730. But several factors may complicate the issue. If currents are applied at right-angles, such as by electrode pads pairs 820/825 and 840/845 in FIG. 8, for example, then the currents may, in general, not be of equal intensity even if the current intensities at the electrodes may be equal. Resulting stimulus intensity may depend, at least in part, on current direction. Accordingly, a stimulus current applied to a nerve fiber may depend, at least in part, on the orientation of the nerve fiber with respect to the stimulus current.

In regions closer to an electrode, current intensity may be higher because current spreading effect may be less near the electrode. If the two currents are not the same magnitude, an interference effect may still be produced, but a resulting waveform may not drop to substantially zero midway between maxima. Thus, an interference effect may still be produced but a depth of modulation of the waveform may be less.

As noted above, for relatively high stimulation efficiency, current may flow substantially parallel to nerve fibers, if there is a single current flow (e.g., applied by one pair of electrode pads) through tissue. If there are two intersecting currents of substantially equal amplitude, relatively high stimulation efficiency may occur along lines substantially midway between the current paths. The reason may be that a net current flow may comprise a vector sum of the two currents.

For example, consider first a situation where two current pathways are at substantially right angles and the current intensities are substantially equal. This is schematically indicated by current vectors 2170. Nerve fibers aligned parallel to one of the current pathways, indicated by arrows 2195, may experience unmodulated stimuli 2172, such as by signals 710 or 720 in FIG. 7, for example. Here, the lengths of the arrows 2190 and 2195 are proportional to the current intensities. In directions at about 45 degrees, there may be no modulation and intensity may be about 30% lower than a case for full modulation (e.g., length of arrows 2195 versus length of arrows 2190). On the other hand, fibers aligned along lines substantially midway between the current paths, indicated by arrows 2190, may experience modulated stimuli 2177 of higher intensity: In the horizontal and vertical directions (indicated by current vectors 2175), net current may be maximum and modulation may be about 100%. Fibers aligned in other directions with respect to currents may experience partially modulated stimuli with a depth of modulation that may depend, at least in part, on the orientation of the fibers with respect to the currents, for example.

In an implementation, nerve fibers aligned in directions which bisect the angle between current pathways (e.g., the vertical current vector indicated in 2180) may experience the greatest stimulation intensity compared to alignments in other directions. Fibers aligned parallel to a direction of individual current flows may experience a lower, but still relatively high, stimulation intensity. The stimulating current need not be modulated. Fibers oriented at some other angle or positioned closer to one electrode may experience a stimulus which is partially modulated. This may be the most common scenario.

In an implementation, nerve fiber firing rates for interferential currents may be much higher than with stimulation using single pulses applied at low frequency. Fibers aligned substantially parallel to the direction of the individual current flows may fire at a rate determined, at least in part, by how far above threshold the local stimulation intensity is. Fibers aligned in directions which substantially bisect the angle between current pathways may fire in relatively high frequency bursts. The bursts of activity may be at the beat frequency and the number of action potentials per burst may depend, at least in part, on how far above threshold the local stimulation intensity may be.

FIG. 22 is a schematic block diagram illustrating a system 2200 for generating electrical signals to apply to a patient for stimulating one or more muscles of the patient, according to an embodiment. For example, system 2200 may comprise two or more individual devices that individually generate such electrical signals. The individual devices may each include a communication port by which the devices may communicate between or among one another to synchronize the electrical signals that each device respectively generates, as explained below. A synchronization technique may be beneficial in that two or more individual devices may be “cascaded” in series or parallel to produce a relatively strong output signal. In another implementation, a synchronization technique may be beneficial in that signals from more than one device may be applied to different portions of a patient, wherein the signals may be synchronized. For example, it may be undesirable if a first signal applied to one part of a patient from one device destructively interferes in the patient with a second signal applied to another part of the patient from another device. Destructive interference may reduce signal strength and/or introduce unintended frequency components in a patient.

Synchronization, on the other hand, may allow two or more signals respectively from different devices to constructively interfere with one another in a patient. Constructive interference may increase signal strength and/or introduce intended frequency components (e.g., of a target signal) in a patient, for example.

System 2200 may be beneficial in that two or more individual devices (e.g., 2210, 2220) may be used for therapy on a patient. From time to time, one may desire to apply stimulation signals to more than one location on a patient at the same time. For example, one pair of electrode pads may apply stimulation signals to a patient's lower left leg, another pair of electrode pads may apply stimulation signals to a patient's upper left leg, and still another pair of electrode pads may apply stimulation signals to a patient's back region. Accordingly, one may use three individual devices to each apply stimulation signals to these portions of the patient. It may be desirable to synchronize stimulation signals (e.g., match their phase) generated by each of the devices so the signals work in unison with one another.

In particular, a first device 2210 may generate a first signal and a second device 2220 may generate a second signal. Device 2210 and 2220 may be similar to device 200 shown in FIG. 2, for example. Device 2210 may include a communication port 2214 and device 2220 may include a communication port 2224. In one implementation, a wire or cable may be used to communicatively connect devices 2210 and 2220 to each other (or to one another, for three or more devices). In another implementation, such communication between or among devices may be implemented by wireless communication. For example, one device may transmit wireless signals to other device(s) to enable synchronization of the stimulation signals that the individual devices respectively generate.

In one embodiment, device 2210 may include an output port 2212 to provide generated first signals to a patient via a first electrode and device 2220 may include an output port 2222 to provide generated second signals to a patient via a second electrode. First and second electrodes may or may not comprise DCT electrode pads, for example. First signals may have substantially the same shape and frequencies as the second signals. Output ports 2212 and 2222 may each be similar to ports 1250 or 1251, for example. In such a case, for example, first signals generated by device 2210 and provided at output port 2212 may be applied to a patient via one pair of electrodes. Accordingly, one electrode may have positive polarity while the other electrode may have negative polarity. (In an implementation, device 2210 may also include an output port (e.g., a second port) to apply the first signals to a patient via a second pair of electrodes. But, for sake of simplicity, such a second pair of electrodes will not be considered in these descriptions of system 2200.) Further, second signals generated by device 2220 and provided at output port 2222 may be applied to the patient via another pair of electrodes. Accordingly, one electrode may have positive polarity while the other electrode may have negative polarity.

First signals may be synchronized with second signals via communication ports 2214 and 2224. For example, the phase of a first signal generated by device 2210 may be synchronized with the phase of a second signal generated by device 2220. Accordingly, therapy applied to a patient using such synchronized first and second signals may be more effective compared to a case where unsynchronized first and second signals are used. For example, unsynchronized first and second signals may interfere with one another in the patient and at least partially cancel out. On the other hand, synchronized first and second signals may interfere with one another to form a superposition of the first and second signals having a magnitude greater than either of the magnitudes of the individual first and second waves.

Any of a number of techniques may be used to synchronize one device with another devices (or devices). For example, if such devices generate output signals using a processor executing code, then such code may include a portion (e.g., subroutine) that generates output signals synchronized to signals of other devices upon or after knowing that such devices exist and are to be synchronized (e.g., via communication ports 2214 and 2224). In another example, if such devices generate output signals using discrete electronic components (e.g., analog electronics), then some of these components may comprise one or more signal generators, gating electronics, and so on. These components may operate based, at least in part, on a system clock. In one implementation, to synchronize output signals of such devices to one another, communication via communication ports, such as 2214 and 2224, for example, may allow system clocks of individual devices to synchronize to one another. This may allow phases of output signals of the individual devices to match one another. In another implementation, to synchronize output signals of such devices to one another, communication via communication ports, such as 2214 and 2224, for example, may allow gating electronics of individual devices to synchronize to one another. This may allow phases of output signals of the individual devices to match one another. Of course, any of a number of techniques may be used for synchronization, and claimed subject matter is not limited to any particular techniques.

In one embodiment, communication ports, such as 2214 and 2224, may allow devices to coordinate with one another, such as to provide one another information regarding signals that each device is generating and/or outputting. Such information may comprise signal information about signals' waveshapes, frequencies, amplitudes, and so on. It may be beneficial for one device to modify characteristics of a signal that it is generating in response to gaining information about characteristics of a signal that another device is generating. For example, two devices may individually generate and output a same particular signal (e.g., for patient therapy). If the two devices are then connected together so as to coordinate with one another (e.g., via ports 2214 and 2224), one device may modify the signal that it generates so that the signal is different from the signal generated by the other device. Modifying a signal may comprise changing a signal's shape, frequencies, amplitude, polarity, duty cycle, phase, and so on. This technique may avoid signal redundancy, for example. In another implementation, two devices may individually generate and output signals that are different from one another. If the two devices are then connected together so as to coordinate with one another (e.g., via ports 2214 and 2224), one device may modify the signal that it generates so that the signal matches (e.g., equals) the signal generated by the other device. Or, one device may modify the signal that it generates so that the signal is modified to generate a particular target signal if superposed with the signal generated by the other device. For example, a first device may generate a first signal comprising signal 1410 and a second device may generate a second signal comprising signal 1510. Upon or after first and second devices are coordinated, second device may modify the second signal so that it comprises signal 1420, so that a superposition of signals 1410 and 1420 comprises a desired target signal 1430, for example. Accordingly, if coordinated devices are used to apply signals (e.g., via electrode pairs from each device) to a patient, the patient may be provided with a target signal. Of course, this is merely an example to help describe embodiments involving coordinated devices. The particular target signal 1430 may not efficiently penetrate skin without using embodiments involving DCT electrode pads, for example. Coordinating devices may generate any signal, and claimed subject matter is not so limited.

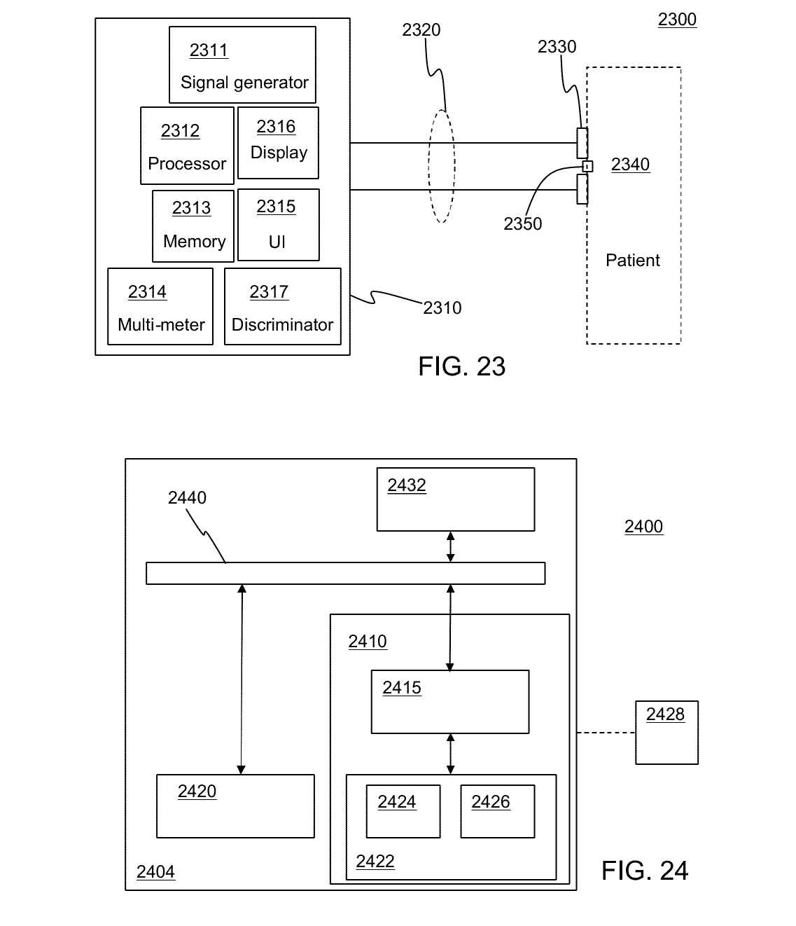

FIG. 23 is a schematic block diagram illustrating a system 2300 for performing a process such as 2000, for example, according to another embodiment. For example, system 2300 may comprise a device 2310, cables 2320, and electrode pads 2330, such as DCT electrode pads. Device 2310 may generate one or more signals that may be applied to a patient 2340 via electrode pads 2330. Device 2310 may include a signal generator 2311 to generate signals having any of a number of parameters, such as waveshape, magnitude, frequency, offset (e.g., from zero volts), and so on. Signal generator 2311 may generate more than one signal at a time, such as a first signal and a second signal depicted in any of FIGS. 14-19, among other examples.

A processor 2312, in addition to or in lieu of signal generator 2311, may be used to, among other things, generate signals provided to electrode pads 2330, which may be electrically connected to patient 2340. Processor 2312 may also evaluate feedback provided by cables 2320 to determine any of a number of parameters. In another implementation, processor 2312 may also evaluate output of detectors 2350 provided via cables 2320, other conductors, or wireless transmission (e.g., from detector 2350 to device 2310). Such detectors may measure one or more parameters representative of a physical condition of subject 2340. For example, such detectors may comprise a blood pressure monitor, blood oxygen level monitor, skin capacitance, skin pH, skin moisture, and so on. Processor 2312 may perform evaluations, calculations, or determinations using parameters measured by multi-meter 2314, for example. Such parameters may include voltage, current, phase shift, and so on.

A discriminator 2317 may decompose or separate a non-sinusoidal signal into two or more individual signals. In one implementation, a voltage signal may include a superposition of any number of individual voltage signals. Current of the voltage signal flowing through patient 2340 may be decomposed by discriminator 2317 so that the current is separated into a number of individual current signals, which may be measured by multi-meter 2314, for example. In one implementation, discriminator 2317 may comprise one or more frequency filters (e.g., low-pass, high-pass, or notch filters, and so on) to perform such signal separation. In another implementation, discriminator 2317 may comprise one or more amplitude filters (e.g., involving resistor networks, diodes, etc.) to perform such signal separation. In yet another implementation, discriminator 2317 may comprise one or more waveshape filters to perform such signal separation. In any case, a composite signal provided to discriminator 2317 (e.g., by cables 2320) may comprise a digital signal. Here, an analog to digital converter (not shown) may be used to convert an analog signal flowing through subject 2340 to a digital signal. Software executed by processor 2312 may be used to identify or distinguish one waveform of one signal from another waveform of another signal in a digital signal. With information from such a processor, discriminator 2317 may separate the separate waveforms and multi-meter 2314 may then measure current or voltage of the separated waveforms. In one implementation, processor 2312 may comprise any of the signal generator 2311, discriminator, or multi-meter, for example.

Device 2310 may further include memory 2313 to store values of parameters measured by multi-meter 2314, or generated by processor 2312 or discriminator 2317, for example. Memory 2313 may also maintain data representative of criteria, rules, or regulations set forth by an agency, group, and so on. Memory 2313 may also store values produced by detectors 2350, for example. Data may comprise tables of values of ranges, maxima, minima, averages, etc. for any of a number of parameters of a signal, such as voltage, current, energy, power, rate of change, and so on. A user interface 2315 may include a keypad, mouse, or touchscreen by which a user may provide operational instructions to device 2310. A display 2316 may display any information to a user, including a graphical representation of a signal provided over cables 2320. Display 2316 may comprise a portion of user interface 2315, and may comprise a touchscreen, touchpad, and so on. Graphical data in display 2316 may be read by processor 2312 in a process of transferring a graphical representation of a signal from display 2316 to digital values stored in memory 2313. Display 2316 may display a graphical representation of a signal that is present on cables 2320 or may display a graphical representation of a virtual signal that is merely proposed so as to not actually be present on cables 2320. Of course, such details of system 2300 are merely examples, and claimed subject matter is not so limited.

FIG. 24 is a schematic diagram illustrating an embodiment of a computing system 2400, for example. Some portions of system 2400 may overlap with some portions of system 2300. System 2400 may be used to perform process 2000, for example. A computing device may comprise one or more processors, for example, to execute an application or other code. A computing device 2404 may be representative of any device, appliance, or machine that may be used to manage memory module 2410. Memory module 2410 may include a memory controller 2415 and a memory 2422. By way of example but not limitation, computing device 2404 may include: one or more computing devices or platforms, such as, e.g., a desktop computer, a laptop computer, a workstation, a server device, or the like; one or more personal computing or communication devices or appliances, such as, e.g., a personal digital assistant, mobile communication device, or the like; a computing system or associated service provider capability, such as, e.g., a database or information storage service provider or system; or any combination thereof.

It is recognized that all or part of the various devices shown in system 2400, and the processes and methods as further described herein, may be implemented using or otherwise including at least one of hardware, firmware, or software, other than software by itself. Thus, by way of example, but not limitation, computing device 2404 may include at least one processing unit 2420 that is operatively coupled to memory 2422 through a bus 2440 and a host or memory controller 2415. Processing unit 2420 is representative of one or more devices capable of performing at least a portion of a computing procedure or process, such as process 2000, for example. By way of example, but not limitation, processing unit 2420 may include one or more processors, microprocessors, controllers, application specific integrated circuits, digital signal processors, programmable logic devices, field programmable gate arrays, and the like, or any combination thereof. Processing unit 2420 may include an operating system to be executed that is capable of communication with memory controller 2415.

An operating system may, for example, generate commands to be sent to memory controller 2415 over or via bus 2440. Commands may comprise read or write commands, for example.

Memory 2422 is representative of any information storage mechanism. Memory may store rules or criteria, signals applied to a subject, output from detectors measuring parameters of a subject, and so on, as explained above. Memory 2422 may include, for example, a primary memory 2424 or a secondary memory 2426. Primary memory 2424 may include, for example, a random access memory, read only memory, etc. While illustrated in this example as being separate from processing unit 2420, it should be understood that all or part of primary memory 2424 may be provided within or otherwise co-located or coupled with processing unit 2420.

Secondary memory 2426 may include, for example, the same or similar type of memory as primary memory or one or more other types of information storage devices or systems, such as a disk drive, an optical disc drive, a tape drive, a solid state memory drive, etc. In certain implementations, secondary memory 2426 may be operatively receptive of, or otherwise capable of being operatively coupled to a computer-readable medium 2428. Computer-readable medium 2428 may include, for example, any medium that is able to store, carry, or make accessible readable, writable, or rewritable information, code, or instructions for one or more of device in system 2400. Computing device 2404 may include, for example, an input/output device or unit 2432.

Input/output unit or device 2432 is representative of one or more devices or features that may be capable of accepting or otherwise receiving signal inputs from a human or a machine, or one or more devices or features that may be capable of delivering or otherwise providing signal outputs to be received by a human or a machine. By way of example but not limitation, input/output device 2432 may include a display, speaker, keyboard, mouse, trackball, touchscreen, etc.

It will, of course, be understood that, although particular embodiments have just been described, claimed subject matter is not limited in scope to a particular embodiment or implementation. For example, one embodiment may be in hardware, such as implemented on a device or combination of devices, for example. Likewise, although claimed subject matter is not limited in scope in this respect, one embodiment may comprise one or more articles, such as a storage medium or storage media that may have stored thereon instructions capable of being executed by a specific or special purpose system or apparatus, for example, to lead to performance of an embodiment of a method in accordance with claimed subject matter, such as one of the embodiments previously described, for example. However, claimed subject matter is, of course, not limited to one of the embodiments described necessarily. Furthermore, a specific or special purpose computing platform may include one or more processing units or processors, one or more input/output devices, such as a display, a keyboard or a mouse, or one or more memories, such as static random access memory, dynamic random access memory, flash memory, or a hard drive, although, again, claimed subject matter is not limited in scope to this example.

The terms, “and” and “or” as used herein may include a variety of meanings that will depend at least in part upon the context in which it is used. Typically, “or” or “and/or” if used to associate a list, such as A, B or C, is intended to mean A, B, and C, here used in the inclusive sense, as well as A, B or C, here used in the exclusive sense.

Embodiments described herein may include machines, devices, engines, or apparatuses that operate using digital signals. Such signals may comprise electronic signals, optical signals, electromagnetic signals, or any form of energy that provides information between locations.

In the description herein, various aspects of claimed subject matter have been described. For purposes of explanation, specific numbers, systems, or configurations may have been set forth to provide a thorough understanding of claimed subject matter. However, it should be apparent to one skilled in the art having the benefit of this disclosure that claimed subject matter may be practiced without those specific details. In other instances, features that would be understood by one of ordinary skill were omitted or simplified so as not to obscure claimed subject matter.

While there has been illustrated and described what are presently considered to be example embodiments, it will be understood by those skilled in the art that various other modifications may be made, and equivalents may be substituted, without departing from claimed subject matter. Additionally, many modifications may be made to adapt a particular situation to the teachings of claimed subject matter without departing from the central concept described herein. Therefore, it is intended that claimed subject matter not be limited to the particular embodiments disclosed, but that such claimed subject matter may also include all embodiments falling within the scope of the appended claims, and equivalents thereof.

### APPENDIX

Information Regarding Tissue and Fourier Frequency Components:

In some embodiments, resistance (e.g., impedance) of biological tissue, such as muscle, fascia, and so on, may depend, at least in part, on presence, location, severity, and/or extent of: inflammation; length-tension relationship of the tissue; forces on the tissue; amount of muscle flexion or extension; injuries; muscle atrophy; hot spots; trigger points; amount of blood or inflammatory fluids present; permeability; elasticity of muscle; and/or cell structure; just to name a few examples. Such conditions of biological tissue (or other biological elements, such as bone, cartilage, ligaments, organs, and so on) may determine, at least in part, how efficiently electrical current of a signal applied via electrodes may travel.

For example, a muscle in a particular amount of flexion may have a resistance (e.g., impedance) different from that of the same muscle in extension. Here, the amount of flexion or extension may determine, at least in part, the amount of resistance. In another example, an injured portion of a muscle may have a resistance different from that of a healthy portion of the same muscle. In yet another example, an inflamed muscle may have a resistance different from that of a non-inflamed muscle. In still another example, a muscle subject to stress (e.g., from muscle imbalance) may have a resistance different from that of the same muscle not subject to such stress. Here, the amount of stress may determine, at least in part, the amount of resistance. In yet another example, a joint with injured ligaments may have a resistance different from that of a healthy joint. In yet another example, an organ with cancer cells may have a resistance different from that of a healthy version of the same organ. Here, the amount of cancer cells may determine, at least in part, the amount of resistance. In yet another example, an amount of blood circulation in a portion of a biological element may have a resistance different from that of another amount of blood circulation in the same portion of the biological element.

In the above examples, resistance of a signal may depend, at least in part, on the frequency of the signal. Thus, to revisit some of the examples above, the resistance of a muscle in a particular amount of flexion for a first signal having a first frequency may be different from that of a second signal having a second frequency. In another example, the resistance of a muscle in a particular amount of extension for a first signal having a first frequency may be different from that of a second signal having a second frequency. In still another example, the resistance of an injured portion of muscle for a first signal having a first frequency may be different from that of a second signal having a second frequency. In still another example, the resistance of an inflamed muscle for a first signal having a first frequency may be different from that of a second signal having a second frequency. In yet another example, the resistance of a muscle subject to a particular amount of stress for a first signal having a first frequency may be different from that of a second signal having a second frequency. In yet another example, the resistance of an organ with cancer cells for a first signal having a first frequency may be different from that of a second signal having a second frequency. In yet another example, the resistance of a biological element having a particular amount of blood circulation for a first signal having a first frequency may be different from that of a second signal having a second frequency.

In the above examples, resistance of a signal may depend, at least in part, on waveshape (e.g., including frequencies) of the signal. Thus, to revisit some of the examples above, the resistance of a muscle in a particular amount of flexion for a first signal having a first waveshape may be different from that of a second signal having a second waveshape. In another example, the resistance of a muscle in a particular amount of extension for a first signal having a first waveshape may be different from that of a second signal having a second waveshape. In still another example, the resistance of an injured portion of muscle for a first signal having a first waveshape may be different from that of a second signal having a second waveshape. In still another example, the resistance of an inflamed muscle for a first signal having a first waveshape may be different from that of a second signal having a second waveshape. In yet another example, the resistance of a muscle subject to a particular amount of stress for a first signal having a first waveshape may be different from that of a second signal having a second waveshape. In yet another example, the resistance of an organ with cancer cells for a first signal having a first waveshape may be different from that of a second signal having a second waveshape. In yet another example, the resistance of a biological element having a particular amount of blood circulation for a first signal having a first waveshape may be different from that of a second signal having a second waveshape.

A device, such as 200, for example, may generate one or more time-dependent signals represented by V(f, t), where f represents frequency and t represents time. Such time dependence may involve cyclically varying wave functions, for example. Such signals may be applied to a patient via electrode pads, for example. A patient may present an impedance Z(f, t) to current I(f′, t) imparted by V(f, t), where distinctions between f and f′ are explained below. It is understood that electrical signal flow may be bi-directional, such as cases where polarity reverses cyclically (e.g., alternating current). Accordingly, even though embodiments may be described as having an output or an input, such designations may be reversed. For example, current I(f′, t) may be provided on one lead and V(f, t) may be provided on another lead, or vise versa. Claimed subject matter is not limited in this respect.

Z(f, t), as indicated by inclusion of the variable for time, t, may be time-dependent. Such time-dependence may account for variable resistance of portions of a patient over time (e.g., of the order of one or two seconds, minutes, or hours). Z(f, t) may include impedances of portions of a patient, for example.

In the example embodiment, V(f, t) may comprise a composite voltage including voltages of two signals: signal 1 and signal 2. These signals may individually include one or more frequency components. For example, in the case of signal 1 comprising a mathematically perfect sine wave having a frequency f₁ and signal 2 comprising a mathematically perfect sine wave having a frequency f₂, V(f, t) may comprise signal 1 with exactly one frequency component and signal 2 with exactly one frequency component, written as

V(f,t)=V₁(f₁,t)+V₂(f₂,t) Eqn. (1)

On the other hand, or in a more general case, signal 1 and/or signal 2 (or their superposition) may comprise non-sinusoidal waves (e.g., square wave, ramp, double- or triple-exponential wave, wave-shape with duty cycle, and so on). In such cases, Fourier components of these waves may comprise a series or sum of frequency terms. For example, signal 1 may have frequency terms f₁ⱼ and signal 2 may have frequency terms f₂ₖ, where j, k=1, 2, 3, . . . . Accordingly, V(f, t) comprising signal 1 and signal 2 with such frequency components, may be written as

V(f,t)=Σ[V₁(f₁ⱼ,t)+V₂(f₂ₖ,t)], Eqn. (2)

where

τV₁(f₁ⱼ,t)=V(f₁₁,t)+V(f₁₂,t)+V(f₁₃,t)+V(f₁₄,t)+ . . . Eqn. (3)

and

ΣV₂(f₂ⱼ,t)=V(f₂₁,t)+V(f₂₂,t)+V(f₂₃,t)+V(f₂₄,t)+ . . . Eqn. (4)

A portion (e.g., skin) of a patient may have an impedance Z(f, t), which is written in bold-face to represent the fact that this impedance may comprise two or more components. For example, Z(f, t) may be written as

Z(f,t)=[Z(f₁ⱼ,t),Z(f₂ₖ,t)], for j,k=1,2,3, . . . Eqn. (5)

where

Z(f₁ⱼ,t)=Z(f₁₁,t),Z(f₁₂,t),Z(f₁₃,t),Z(f₁₄,t), Eqn. (6)

and

Z(f₂ⱼ,t)=Z(f₂₁,t),Z(f₂₂,t),Z(f₂₃,t),Z(f₂₄,t), Eqn. (7)

Here, Z(f₁ⱼ, t) may represent a set of impedances for signal 1 and Z(f₂ⱼ, t) may represent a set of impedances for signal 2. For example, as discussed above, impedances of various biological elements of a patient may be frequency-dependent. Accordingly, impedance terms for individual frequencies may correspond to individual voltage terms of the same individual frequencies. In one implementation, difference of capacitive reactances (e.g., the imaginary component of impedance) of electrode-skin interfaces between that of signal 1 and that of signal 2 may be negligible and ignored in some applications. For example, capacitive reactances of electrode-skin interfaces for signal 1 and for signal 2 may be substantially equal or similar (e.g., different by less than about a few percent) for some ranges of frequencies. For a numerical example, capacitive reactance of electrode-skin interfaces for signal 1 having a first order frequency (e.g., a largest term in a Fourier series of terms) of 10,000 Hz may be less than 2% different from a capacitive reactance for signal 2 having a first order frequency of 12,000 Hz.

In another implementation, difference of capacitive reactances of electrode-skin interfaces between that of signal 1 and that of signal 2 may be accounted for by maintaining a table of values (or other format of such information) of capacitive reactances of electrode-skin interfaces for a plurality of frequencies for particular subjects or for types of subjects. For example, subjects may have particular skin conditions (e.g., having various values of pH, moisture content, and so on). For example, for a particular subject (or particular class of subjects), a table of values may comprise values for capacitive reactance of electrode-skin interfaces for different signal frequencies. Thus, it may be desirable to discount differences in capacitive reactance of electrode-skin interfaces between that of signal 1 and signal 2.

In the example embodiment, I(f′, t) may comprise a composite current including currents of signal 1 and signal 2. Fourier components of these signals may comprise a series or sum of frequency terms. For example, signal 1 may have frequency terms f′₁ⱼ and signal 2 may have frequency terms f′₂ₖ, where j, k=1, 2, 3, . . . . Accordingly, I(f′, t) comprising currents of signal 1 and signal 2 with such frequency components, may be written as

I(f′,t)=Σ[I₁(f′₁ⱼ,t)+I₂(f′₂ₖ,t)] Eqn. (8)

where

ΣI₁(f′₁ⱼ,t)=I(f′₁₁,t)+I(f′₁₂,t)+I(f′₁₃,t)+I(f′₁₄,t)+ . . . Eqn. (9)

and

ΣI₂(f′₂ⱼ,t)=I(f′₂₁,t)+I(f′₂₂,t)+I(f′₂₃,t)+I(f′₂₄,t)+ . . . Eqn. (10)

Fourier components of these current waves may comprise a series or sum of frequency terms that may be different from corresponding terms in ΣV₁(f₁ⱼ, t) and ΣV₂(f₂ⱼ, t). For example, impedances of a patient may shift the phases of currents of the different frequencies with respect to the phases of the corresponding voltages. Thus, frequencies of some Fourier terms of current may be altered from those of the voltage based, at least in part, on frequency-dependent impedances (e.g., capacitive or inductive reactances). (In other words, a shape of a current wave may be distorted from that of the voltage wave by frequency-dependent impedances: Thus, frequencies of Fourier terms of the current may be different from those of the voltage to account for the wave-shape distortion. If impedance were not frequency-dependent, for example, then there may be no phase shifts between voltage and current Fourier terms.)

Using Ohm's Law for signal 1,

Z(f₁ₘ,t)=V(f₁ₘ,t)/I(f′₁ₘ,t), for m=1,2,3, . . . Eqn. (11)

and for signal 2,

Z(f₂ₘ,t)=V(f₂ₘ,t)/I(f′₂ₘ,t), for m=1,2,3, . . . Eqn. (12)

Of course, such details of voltage, current, and impedance are merely examples, and claimed subject matter is not so limited.

What is claimed is:

1. A method for providing electrical stimulation to one or more muscles in a patient, the method comprising:
generating a first electrical signal having a time-varying waveform;
generating a second electrical signal having a time-varying waveform, wherein a sum of said first electrical signal and said second electrical signal comprises a target electrical signal having a waveform to generate substantial motor movement of said one or more muscles in said patient; and
applying said first electrical signal via a first electrode and said second electrical signal via a second electrode to a particular location on said patient, wherein said first electrode and said second electrode are on a single electrode pad.

2. The method of claim 1, wherein said applying further comprises placing said single electrode pad on skin of said patient so that said first electrode and said second electrode are electrically separated by capacitance of said skin based, at least in part, on electrical properties of dermis and subcutis of said skin.

3. The method of claim 1, wherein said applying further comprises placing said single electrode pad on skin of said patient so that said first electrical signal and said second electrical signal superpose below said skin to form said target electrical signal.

4. The method of claim 3, wherein said target electrical signal has a frequency based, at least in part, on a frequency of said first electrical signal and a frequency of said second electrical signal.

5. The method of claim 1, wherein said frequency of said first electrical signal and said frequency of said second electrical signal are greater than about 2000 Hertz and a frequency of said target electrical signal is less than about 600 Hertz.

6. The method of claim 1, wherein said single electrode pad comprises a positive electrode pad of a circuit generating said first and said second electrical signals, and further comprising:
applying said first electrical signal via a first negative electrode and said second electrical signal via a second negative electrode to another particular location on said patient, wherein said first negative electrode and said second negative electrode are on a single negative electrode pad of said circuit.

7. The method of claim 6, wherein said one or more muscles are electrically between said particular location and said another particular location.

8. The method of claim 1, wherein said first electrical signal and said second electrical signal are applied to said patient while said patient is substantially moving.

9. The method of claim 1, wherein said particular location on said patient and at least one of said one or more muscles in said patient are apart by more than about one inch.

10. The method of claim 1, wherein said first electrical signal comprises a first set of pulses that are 180 degrees out of phase with a second set of pulses in said second electrical signal.

11. The method of claim 1, wherein said target electrical signal comprises a summed periodic-exponential signal.

12. An apparatus for providing electrical stimulation to one or more muscles in a patient, said apparatus comprising:
an electrical circuit to generate a first electrical signal having a time-varying waveform and a second electrical signal having a time-varying waveform, wherein a sum of said first electrical signal and said second electrical signal comprises a target electrical signal having a waveform to stimulate movement of said one or more muscles in said patient; and
an output port to provide said first output signal and said second output signal to said patient via a pair of double-capacitive transcutaneous electrode pads.

13. The apparatus of claim 12, wherein said output port is configured to apply said first electrical signal to first electrodes of individual ones of said pair of double-capacitive transcutaneous electrode pads and to apply said second electrical signal to second electrodes of said individual ones of said pair of double-capacitive transcutaneous electrode pads.

14. The apparatus of claim 12, wherein each of said double-capacitive transcutaneous electrode pads comprises:
a first electrode and a second electrode disposed on a single pad that electrically interacts with dermis and subcutis of skin of said patient.

15. The apparatus of claim 12, wherein each of said double-capacitive transcutaneous electrode pads comprises a first electrode and a second electrode electrically separated by capacitance of said skin based, at least in part, on electrical properties of dermis and subcutis of skin of said patient.

16. The apparatus of claim 12, wherein said target electrical signal has a frequency based, at least in part, on a frequency of said first electrical signal and a frequency of said second electrical signal.

17. The apparatus of claim 12, wherein said frequency of said first electrical signal and said frequency of said second electrical signal are greater than about 2000 Hertz and a frequency of said target electrical signal is less than about 600 Hertz.

18. The apparatus of claim 12, wherein said first electrical signal comprises a first set of pulses that are 180 degrees out of phase with a second set of pulses in said second electrical signal.

19. The apparatus of claim 12, wherein said target electrical signal comprises a summed periodic-exponential signal.

20. An apparatus for providing electrical stimulation to one or more muscles in a patient, said apparatus comprising:
means for generating a first time-varying signal and a second time-varying signal;
means for applying said first time-varying signal and said second time-varying signal to a patient;
means for summing said first time-varying signal and said second time-varying signal below skin at a particular location of said patient to produce a triple exponential signal, said triple exponential signal comprising:
a feature pulse for stimulating movement of said one or more muscles and having a frequency below about 1000 Hz; and
a carrier pulse having a frequency greater than about 1000 Hz, wherein said carrier pulse is amplitude modulated.
